SEQUENCE LISTING (43 sequences)

SEQ ID NO: 1 - protein, 115 aa, synthetic construct
  REGION 1..115: note = Synthetic Construct
QSVEESGGGLVTPGTPLTLTCTVSGIDLSRYAMSWVRQAPGKGLEWIGIFGSLGGIFYASWAKGRFTISKTSPTTVDLKITSPTTEDTATYFCARMPYTTDRDFWGPGTLVTVSS

SEQ ID NO: 2 - protein, 110 aa, synthetic construct
  REGION 1..110: note = Synthetic Construct
DIVMTQTPSSTSAAVGGTVTITCQASQSVANNNYLKWYQQKRGQPPKQLIYSVSTLASGVPSRFKGSGSGTQFTLTISDLECDDAATYYCSGYFNNNIGAFGGGTKLEIK

SEQ ID NO: 3 - protein, 5 aa, synthetic construct
  REGION 1..5: note = Synthetic Construct
RYAMS

SEQ ID NO: 4 - protein, 15 aa, synthetic construct
  REGION 1..15: note = Synthetic Construct
IFGSLGGIFYASWAK

SEQ ID NO: 5 - protein, 9 aa, synthetic construct
  REGION 1..9: note = Synthetic Construct
MPYTTDRDF

SEQ ID NO: 6 - protein, 13 aa, synthetic construct
  REGION 1..13: note = Synthetic Construct
QASQSVANNNYLK

SEQ ID NO: 7 - protein, 7 aa, synthetic construct
  REGION 1..7: note = Synthetic Construct
SVSTLAS

SEQ ID NO: 8 - protein, 10 aa, synthetic construct
  REGION 1..10: note = Synthetic Construct
SGYFNNNIGA

SEQ ID NO: 9 - protein, 115 aa, synthetic construct
  REGION 1..115: note = Synthetic Construct
QTVKESGGRLVTPGTPLTLTCTVSGIDLTTYAMTWVRQAPGKGLEWIGIIASSDDTNYASWAKGRFTIYKTSSTTVDLSITSPTTEDTATYFCARMPYTTDRDIWGPGTLVTVSS

SEQ ID NO: 10 - protein, 110 aa, synthetic construct
  REGION 1..110: note = Synthetic Construct
DVVMTQTPSSVSAAVGGTVTISCQASQSVALNSYLKWYQQKPGQPPKQLIYSVSTLASGVPSRFKGSGSGTQFTLTISDLECDDAATYYCSGYFNGNIGAFGAGTKLEIK

SEQ ID NO: 11 - protein, 5 aa, synthetic construct
  REGION 1..5: note = Synthetic Construct
TYAMT

SEQ ID NO: 12 - protein, 15 aa, synthetic construct
  REGION 1..15: note = Synthetic Construct
IIASSDDTNYASWAK

SEQ ID NO: 13 - protein, 9 aa, synthetic construct
  REGION 1..9: note = Synthetic Construct
MPYTTDRDI

SEQ ID NO: 14 - protein, 13 aa, synthetic construct
  REGION 1..13: note = Synthetic Construct
QASQSVALNSYLK

SEQ ID NO: 15 - protein, 7 aa, synthetic construct
  REGION 1..7: note = Synthetic Construct
SVSTLAS

SEQ ID NO: 16 - protein, 10 aa, synthetic construct
  REGION 1..10: note = Synthetic Construct
SGYFNGNIGA

SEQ ID NO: 17 - protein, 438 aa, synthetic construct
  REGION 1..438: note = Synthetic Construct
QSVEESGGGLVTPGTPLTLTCTVSGIDLSRYAMSWVRQAPGKGLEWIGIFGSLGGIFYASWAKGRFTISKTSPTTVDLKITSPTTEDTATYFCARMPYTTDRDFWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 18 - protein, 214 aa, synthetic construct
  REGION 1..214: note = Synthetic Construct
DIVMTQTPSSTSAAVGGTVTITCQASQSVANNNYLKWYQQKRGQPPKQLIYSVSTLASGVPSRFKGSGSGTQFTLTISDLECDDAATYYCSGYFNNNIGAFGGGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 19 - protein, 438 aa, synthetic construct
  REGION 1..438: note = Synthetic Construct
QTVKESGGRLVTPGTPLTLTCTVSGIDLTTYAMTWVRQAPGKGLEWIGIIASSDDTNYASWAKGRFTIYKTSSTTVDLSITSPTTEDTATYFCARMPYTTDRDIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 20 - protein, 214 aa, synthetic construct
  REGION 1..214: note = Synthetic Construct
DVVMTQTPSSVSAAVGGTVTISCQASQSVALNSYLKWYQQKPGQPPKQLIYSVSTLASGVPSRFKGSGSGTQFTLTISDLECDDAATYYCSGYFNGNIGAFGAGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 21 - protein, 438 aa, synthetic construct
  REGION 1..438: note = Synthetic Construct
QSVEESGGGLVTPGTPLTLTCTVSGIDLSRYAMSWVRQAPGKGLEWIGIFGSLGGIFYASWAKGRFTISKTSPTTVDLKITSPTTEDTATYFCARMPYRTDRDFWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 22 - protein, 438 aa, synthetic construct
  REGION 1..438: note = Synthetic Construct
QSVEESGGGLVTPGTPLTLTCTVSGIDLSRRAMSWVRQAPGKGLEWIGIFGSLGGIFYASWAKGRFTISKTSPTTVDLKITSPTTEDTATYFCARMPYTTDRDFWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 23 - protein, 438 aa, synthetic construct
  REGION 1..438: note = Synthetic Construct
QSVEESGGGLVTPGTPLTLTCTVSGIDLSRRAMSWVRQAPGKGLEWIGIFGSLGGIFYASWAKGRFTISKTSPTTVDLKITSPTTEDTATYFCARMPYRTDRDFWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 24 - protein, 438 aa, synthetic construct
  REGION 1..438: note = Synthetic Construct
QTVKESGGRLVTPGTPLTLTCTVSGIDLTTYAMTWVRQAPGKGLEWIGIIASSDDTNYASWAKGRFTIYKTSSTTVDLSITSPTTEDTATYFCARMPYRTDRDIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 25 - protein, 438 aa, synthetic construct
  REGION 1..438: note = Synthetic Construct
QTVKESGGRLVTPGTPLTLTCTVSGIDLTTRAMTWVRQAPGKGLEWIGIIASSDDTNYASWAKGRFTIYKTSSTTVDLSITSPTTEDTATYFCARMPYTTDRDIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 26 - protein, 438 aa, synthetic construct
  REGION 1..438: note = Synthetic Construct
QTVKESGGRLVTPGTPLTLTCTVSGIDLTTRAMTWVRQAPGKGLEWIGIIASSDDTNYASWAKGRFTIYKTSSTTVDLSITSPTTEDTATYFCARMPYRTDRDIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 27 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 28 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 29 - protein, 434 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 30 - protein, 402 aa, Mus musculus
MADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 31 - protein, 373 aa, Mus musculus
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEANGRTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

SEQ ID NO: 32 - protein, 6 aa, synthetic construct
  REGION 1..6: note = Synthetic Construct
LFFEVD

SEQ ID NO: 33 - protein, 6 aa, synthetic construct
  REGION 1..6: note = Synthetic Construct
GDLESD

SEQ ID NO: 34 - protein, 9 aa, Homo sapiens
RCLHNFLTD

SEQ ID NO: 35 - protein, 9 aa, Homo sapiens
EDDLFFEAD

SEQ ID NO: 36 - protein, 9 aa, Homo sapiens
EDDENLESD

SEQ ID NO: 37 - protein, 9 aa, Mus musculus
GKQLSLLSD

SEQ ID NO: 38 - protein, 9 aa, Mus musculus
ENDLFFEVD

SEQ ID NO: 39 - protein, 9 aa, Mus musculus
EENGDLESD

SEQ ID NO: 40 - protein, 9 aa, Mus musculus
DLPRNMEAD

SEQ ID NO: 41 - protein, 6 aa, synthetic construct
  REGION 1..6: note = Synthetic Construct
EAYVHD

SEQ ID NO: 42 - protein, 9 aa, Homo sapiens
WDNEAYVHD

SEQ ID NO: 43 - protein, 9 aa, Mus musculus
DDDNLLVCD

(19) United States
(12) Patent Application Publication
(10) Pub. No.: US 2023/0399417 A1
(43) Pub. Date: Dec. 14, 2023

(54) ANTI-CLEAVED ICASPASE SUBSTRATE ANTIBODIES AND METHODS OF USE

(71) Applicant: Genentech, Inc., South San Francisco, CA (US)

(72) Inventors: Christopher W. DAVIES, Oakland, CA (US); Nobuhiko KAYAGAKI, Millbrae, CA (US); James T. KOERBER, San Mateo, CA (US)

(21) Appl. No.: 18/301,169

(22) Filed: Apr. 14, 2023

Related U.S. Application Data
(63) Continuation of application No. PCT/US2021/071871, filed on Oct. 14, 2021.
(60) Provisional application No. 63/093,026, filed on Oct. 16, 2020.

(51) Int. Cl. C07K 16/40; C12N 15/10

(52) U.S. Cl. CPC C07K 16/40; C12N 15/1044; C07K 2317/565

(57) ABSTRACT

Provided herein are antibodies that bind to cleaved inflammatory caspase (iCaspase) substrates and methods of screening for such antibodies. Also provided herein are detection methods using such antibodies for detecting a cleaved iCaspase substrate.

### CROSS-REFERENCE TO RELATED APPLICATIONS

This application is a continuation of International Application No. PCT/US2021/071871 having an international filing date of Oct. 14, 2021, which claims priority benefit to U.S. Provisional Application No. 63/093,026, filed Oct. 16, 2020, the contents of which are hereby incorporated herein by reference in their entirety.

### REFERENCE TO AN ELECTRONIC SEQUENCE LISTING

The contents of the electronic sequence listing (146392051301seqlist.xml; Size: 55,306 bytes; and Date of Creation: Mar. 31, 2023) are herein incorporated by reference in their entirety.

### FIELD OF THE INVENTION

The present invention relates to antibodies that binds to a cleaved inflammatory caspase substrate and methods of using the same.

### BACKGROUND OF THE INVENTION

Inflammasomes are a component of the innate immune system that sense a number of pathogen and host-damage signals, and, in response, initiate a signaling cascade triggering inflammatory cell death or pyroptosis. The inflammatory caspases are the key effectors of this process through the cleavage and activation of gasdermin D. Further, an inflammatory caspase (caspase-1) activates the pro-inflammatory interleukins IL-1β and IL-18, via proteolysis. Therefore, the inflammatory caspases and their substrates are important aspects of the innate immune system.

Relative to the well-studied apoptotic caspases, knowledge of the identities of substrates of the inflammatory caspases remains limited. A greater understanding of these substrates may shed light on the biological functions of the inflammatory caspases. Further, substrates of inflammatory caspase may serve as blood-based biomarkers of inflammasome activation.

Accordingly, there exists a need in the art for means of targeting cleaved substrates of the inflammatory caspases. In particular, there exists a need for antibodies that specifically bind peptides with a similar degenerate recognition motif as the inflammatory caspases, without recognizing the canonical apoptotic caspase recognition motif.

### BRIEF SUMMARY

In one aspect, the present invention provides an antibody that binds to a cleaved inflammatory caspase (iCaspase) substrate, wherein the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

In some embodiments, P4 is a hydrophobic amino acid

In some embodiments, P4 is selected from the group consisting of W, F, L, I, P, and Y.

In some embodiments, P4 is W or I.

In some embodiments, P3 is selected from the group consisting from Q and E and P2 is selected from the group consisting of S and T.

In some embodiments, the antibody binds to a cleaved substrate of Caspase 1, Caspase 4, Caspase 5, or Caspase 11.

In some embodiments, the antibody is a rabbit, rodent, or goat antibody.

In some embodiments, the antibody is a full length antibody, a Fab fragment, or an scFv.

In some embodiments, the antibody is conjugated to a label.

In some embodiments, the label is selected from the group consisting of biotin, digoxigenin, and fluorescein.

In some embodiments, the antibody comprises a variable heavy chain (VH) and a variable light chain (VL), wherein the antibody comprises a CDRH1, a CDRH2, and a CDRH3 of a VH chain comprising the amino acid sequence set forth in SEQ ID NO: 1 and a CDRL1, CDRL2, and CDRL3 of a VL chain comprising the amino acid sequence set forth in SEQ ID NO: 2.

In some embodiments, the antibody comprises a CDRH1 amino acid sequence set forth in SEQ ID NO: 3; a CDRH2 amino acid sequence set forth in SEQ ID NO: 4; a CDRH3 set forth in SEQ ID NO:5; a CDRL1 amino acid sequence set forth in SEQ ID NO: 6; a CDRL2 amino acid sequence set forth in SEQ ID NO:7; and a CDRL3 amino acid sequence set forth in SEQ ID NO:8.

In some embodiments, the antibody comprises a VH chain amino acid set forth in SEQ ID NO: 1 and a VL chain amino acid set forth in SEQ ID NO:2.

In some embodiments, the antibody comprises a variable heavy chain (VH) and a variable light chain (VL), wherein the antibody comprises a CDRH1, a CDRH2, and a CDRH3 of a VH chain comprising the amino acid sequence set forth in SEQ ID NO: 9 and a CDRL1, CDRL2, and CDRL3 of a VL chain comprising the amino acid sequences set forth in SEQ ID NO: 10.

In some embodiments, the antibody comprises a CDRH1 amino acid sequence set forth in SEQ ID NO: 11; a CDRH2 amino acid sequence set forth in SEQ ID NO: 12; a CDRH3 amino acid sequence set forth in SEQ ID NO:13; a CDRL1 amino acid sequence set forth in SEQ ID NO: 14; a CDRL2 amino acid sequence set forth in SEQ ID NO:15; and a CDRL3 amino acid sequence set forth in SEQ ID NO:16.

In some embodiments, the antibody comprises a VH chain amino acid sequence set forth in SEQ ID NO: 9 and a VL chain amino acid sequence set forth in SEQ ID NO:10.

In another aspect, a nucleic acid encoding the antibody of any one of the above embodiments is provided.

In another aspect, a host cell comprising the nucleic acid of paragraph [0022] is provided.

In another aspect, the present invention provides a method of screening for an antibody that binds to a cleaved iCaspase substrate, wherein the antibody specifically binds to a peptide comprising the amino acid sequence P4-P3-P2-D at the C-terminus of the peptide, wherein the antibody does not bind to peptides comprising the amino acid sequence D-X-X-D at the C-terminus, wherein X is any amino acid, comprising
i) providing an antibody library;
ii) positively selecting antibodies that bind to a peptide comprising the amino acid sequence P4-P3-P2-D motif at the C-terminus; and
iii) negatively selecting antibodies that bind to a peptide comprising the amino acid sequence D-X-X-D at the C-terminus,
thereby producing an antibody that specifically binds to a peptide comprising the amino acid P4-P3-P2-D at the C-terminus, and does not bind to peptides comprising the amino acid sequence D-X-X-D at the C-terminus.

In some embodiments, the method further comprises negatively selecting antibodies that bind to a peptide comprising the amino acid sequence E-X-X-D at the C-terminus.

In some embodiments, negatively selecting antibodies that bind to a peptide comprising the amino acid sequence E-X-X-D at the C-terminus is performed simultaneously with step iii).

In some embodiments, negatively selecting antibodies that bind to a peptide comprising the amino acid sequence E-X-X-D at the C-terminus is performed before or after step iii).

In some embodiments, P4 is a hydrophobic amino acid.

In some embodiments, the library is a phage library or a yeast library.

In some embodiments, the library is produced by immunizing a mammal with a peptide library comprising peptides comprising the following sequences W-P3-P2-D, Y-P3-P2-D, I-P3-P2-D, and L-P3-P2-D, wherein P3 is an equimolar mixture of E, V, and Q and P2 is an equimolar mixture of H, S, and T, wherein the mammal produces antibodies to the peptides.

In some embodiments, the mammal is a rabbit or a mouse.

In some embodiments, steps ii)-iii) are repeated two or more times.

In another aspect, an antibody produced by the method of paragraphs [0024]-[0032] is provided.

In another aspect, the present invention provides a method of detecting cleavage of an iCaspase substrate in a sample comprising
i) contacting the sample with an anti-cleaved iCaspase substrate antibody, and
ii) detecting a cleaved iCaspase substrate
wherein the anti-cleaved iCaspase substrate antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

In some embodiments, P4 is a hydrophobic amino acid.

In some embodiments, the cleaved iCaspase substrate is detected using a secondary antibody that binds to the anti-cleaved iCaspase substrate antibody.

In another aspect, the present invention provides a method of enriching cleaved iCaspase substrates in a sample comprising a mixture of polypeptides
i) contacting the sample with an anti-cleaved iCaspase substrate antibody; and
ii) selecting antibody-bound polypeptides from the sample,
wherein the anti-cleaved iCaspase substrate antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

In some embodiments, the method further comprises detecting the selected antibody-bound polypeptides.

In some embodiments, the antibody-bound polypeptides are detected by protein sequencing.

In another aspect, the present invention provides a library of cleaved iCaspase substrates produced by the method of paragraph [0037].

In another aspect, the present invention provides a kit for detecting a cleaved iCaspase substrate in a sample comprising an anti-cleaved iCaspase substrate antibody and instructions for use, wherein the anti-cleaved iCaspase substrate antibody specifically binds to a peptide comprising the amino acid sequence P4-P3-P2-D at the C-terminus of the peptide, wherein P4 is a hydrophobic amino acid, wherein the antibody does not bind to peptides comprising the amino acid sequence D-X-X-D at the C-terminus, wherein X is any amino acid.

In some embodiments, the anti-cleaved iCaspase substrate antibody is conjugated to a label.

### BRIEF DESCRIPTION OF THE FIGURES

FIG. 1A provides the design of two different degenerate peptide libraries used in the rabbit immunization strategy to generate antibodies that bind to cleaved iCaspase substrates. The identity of the amino acid residues at each of four positions (from N- to C-terminus, P4, P3, P2, and P1) is shown, and the relative proportion of each residue at each position is indicated by its height. In library A (top row) the library sequence is W/YxxD, and in library B (bottom row) the library sequence is I/LxxD, where P3 is an equimolar mixture of E, V, and Q and P2 is an equimolar mixture of H, S, and T. FIG. 1B shows sequences of C-terminal peptide products in known inflammatory caspase substrates generated upon proteolysis in humans (from top to bottom row of the table, SEQ ID NOs: 34, 35, 42, and 36) and mice (from top to bottom row of the table, SEQ ID NOs: 37, 38, 43, 39, and 40. FIG. 1C provides data from an ELISA testing the ability of purified polyclonal sera to bind to the WxxD (black bars) or IxxD (gray bars) libraries in comparison to a DxxD library (checkerboard bars) and a BSA (diagonally striped bars) control. The identity of the serum is shown on the x-axis, the optical density at 650 nm is shown on the y-axis, n=3, and the error bars indicate the standard deviation. FIG. 1D provides data from an ELISA measuring the ability of purified polyclonal sera to bind to peptides corresponding to proteolysis products generated in known inflammatory caspase substrates (hGsdmd, black bars; mGsdmd, gray bars; hIL1β.A, diagonally striped bars; mIL1β.A, checkerboard bars; h/mIL18, horizontally striped bars) in comparison to streptavidin (control) (white bars). The identity of the serum is shown on the x-axis, the optical density at 650 nm is shown on the y-axis, and the error bars indicate the standard deviation.

FIG. 2 shows western blots of stimulated bone marrow derived macrophages (BMDMs) with purified polyclonal sera from immunized rabbits. As indicated above each blot, BMDMs were either control samples (CON), or samples stimulated with LPS and cholera toxin B (LPS+CTB), or ATP. The identity of the sera used is indicated below each blot, including, from left to right, Rabbit 35, Rabbit 37, Rabbit 38, Rabbit 39, Rabbit 40, Rabbit 44, Rabbit 45, and Rabbit 46. Examples of bands unique to the stimulated BMDM lysates compared to control lysates (i.e., pyroptosis-specific bands) are indicated by arrows. All ELISAs were performed in triplicate with errors bars representing the standard deviation.

FIG. 3A shows a schematic summary of the rabbit immune phage workflow to generate monoclonal antibodies with inflammatory caspase-like specificity. FIG. 3B provides data from an ELISA measuring the ability of antibodies CJ11 and CJ2 to bind (from left to right for each antibody) WxxD, IxxD, WxxD-NH₂, IxxD-NH₂, DxxD, or BSA peptides. The identity of the antibody is shown on the x-axis with CJ11 at left and CJ2 at right, the optical density at 650 nm is shown on the y-axis, and the error bars indicate the standard deviation. FIG. 3C provides data from an ELISA measuring the ability of antibodies CJ11 and CJ2 to bind (from left to right for each antibody) hGsdmd, mGsdmd, hIL1β.A, mIL1β.A, hIL1β.B, mIL1β.B, h/mIL18, or streptavidin. The identity of the antibody is shown on the x-axis with CJ11 at left and CJ2 at right, the optical density at 650 nm is shown on the y-axis, and the error bars indicate the standard deviation. FIG. 3D provides data from an ELISA measuring the ability of antibodies CJ11 and CJ2 to bind (from left to right for each antibody) Gsdmd, Gsdmd with an additional glycine residue (+G), Casp11, Casp11 with an additional alanine residue (+A), or streptavidin. The identity of the antibody is shown on the x-axis with CJ11 at left and CJ2 at right, the optical density at 650 nm is shown on the y-axis, and the error bars indicate the standard deviation.

FIG. 4A shows specificity profiles of antibody CJ11 as determined by phage display. Phage display selections against both antibodies were performed using two degenerate peptide libraries: X₁₂—COOH (top profile) and X₉D-COOH (bottom profile), where X is any of the twenty amino acids. FIG. 4B shows specificity profiles of antibody CJ2 as determined by phage display. Phage display selections against both antibodies were performed using two degenerate peptide libraries: X₁₂—COOH (left profile) and X₉D-COOH (right profile), where X is any of the twenty amino acids.

FIG. 5A provides a view of the co-crystal structure of the IL-1β+CJ11 complex. The mouse IL-1β peptide (₂₁LFFEVD₂₆) (SEQ ID NO: 32) is shown as a stick representation with the positions of the individual amino acid residues of the peptide labeled, and the light chain (LC) and heavy chain (HC) of CJ11 are labeled and shown as cartoon ribbon representations. FIG. 5B shows a close-in view of the IL-1β Asp26 interaction with CJ11. The IL-1β peptide is shown in the foreground in dark gray, with residues V25 and D26 labeled; all other residues shown are CJ11 residues. Hydrogen bond and ionic interactions are shown as dotted lines, water molecules are shown as spheres, and residues indicated by an asterisk (*) indicate an interaction with the protein backbone. FIG. 5C shows a view of interactions across the IL-1β-CJ11 complex. The IL-1β peptide is shown in the foreground in dark gray, with residues L21, F22, F23, E24, V25, and D26 labeled; all other residues shown are CJ11 residues. Hydrogen bond and ionic interactions are shown as dotted lines, water molecules are shown as spheres, and residues indicated by an asterisk (*) indicate an interaction with the protein backbone. FIG. 5D shows a superposition of the IL-18+CJ11 complex with the IL-1β+CJ11 complex, with the IL18 peptide (₃₀GDLESD₃₅) (SEQ ID NO: 33) residues labeled, and both peptides shown as stick representations. IL-18 and IL-1β are labeled. FIG. 5E shows the IL-1β+CJ11 complex with Fo-Fe electron density map contoured at 3.0 σ and calculated using diffraction data extending to 2 Å resolution. The IL-1β peptide is shown as a stick representation with the positions of the individual amino acid residues of the peptide labeled, and the light chain (LC) and heavy chain (HC) of CJ11 are labeled and shown as cartoon ribbon representations. FIG. 5F shows the IL-18+CJ11 complex with a Fo-Fc electron density map contoured at 1.75 σ and calculated using diffraction data extending to 1.7 Å resolution. The IL-18 peptide is shown as a stick representation with the positions of the individual amino acid residues of the peptide labeled, and the light chain (LC) and heavy chain (HC) of CJ11 are labeled and shown as cartoon ribbon representations.

FIG. 6A shows immunoblots of lysates from HEK293 cells overexpressing full-length (F) or cleaved (Cl) forms of FLAG-tagged caspase substrates (IL-1β, IL-18, caspase-11, and GSDMD, as indicated from left to right above the blots). Immunoprecipitations were performed with either CJ11 (right) or anti-FLAG monoclonal antibody (left), followed by anti-FLAG Westerns for detection. FIG. 6B shows anti-CJ11 immunoblots of CJ11 immunoprecipitations from wild-type (WT, left lanes) or CASP1 knockout (right lanes) immortalized mouse macrophages. Macrophages were tested either stimulated with cytosolic LPS (+) or not (−).

FIG. 7 shows Western blots of EA.hy926 cell lysate (left) and supernatant (right) used for CJ11 immunoprecipitation-mass spectrometry experiments. The lysate and supernatants were probed with an anti-caspase-4 antibody (top), anti-Gsdmd antibody (center), and CJ11 (bottom).

FIG. 8A shows a volcano plot showing CJ11-immunoprecipitated substrates from EA.hy926 cells, as determined by mass spectrometry. The x-axis shows the log₂-transformed fold change of each substrate, and the y-axis shows the −log₁₀ transformed adjusted p-value. Peptides identified in the lysate and supernatant are shown as circles and triangles, respectively, and the position of Gsdmd on the plot is labeled. FIG. 8B shows a sequence logo from CJ11-immunoprecipitated peptides enriched at least two-fold upon LPS treatment. Amino acids after the Asp (labeled 1-6) correspond to the sequence from the native full-length protein. FIG. 8C shows a portion of Gene Ontology analysis of CJ11-immunoprecipitated substrates. FIG. 8D shows a substrate interaction network for spliceosome components that were immunoprecipitated by CJ11. FIG. 8E shows a Western blot analysis of GSDMD (left) and caspase-7 (right) in wild-type vs. CASP4 knockout EA.hy926 cells that were stimulated with LPS (+) or not (−). Full-length GSDMD and caspase-7 are indicated with black arrows and cleaved forms are indicated with open arrows.

FIG. 9 shows structural alignment of caspase-1, -4, and -11 substrate-binding pockets. The WEHD-aldehyde substrate (PDB 1IBC) is labeled. Key residues comprising the binding pockets for the substrate are shown as sticks. Caspase-1 (PDB 1IBC), caspase-4 (PDB 6KMZ), and caspase-11 (PDB 6KMV) were used to generate the alignment.

FIG. 10 shows a Western blot using CJ11 to detect caspase substrates. TRAIL-stimulated cells (+) were lysed and subjected to immunoprecipitation and subsequent Western blot.

### DETAILED DESCRIPTION

### I. Definitions

“Hydrophobic amino acid” as used herein means tryptophan, phenylalanine, tyrosine, isoleucine, leucine, valine, methionine, or alanine.

“iCaspase” as used herein is an inflammatory caspase. As described, below iCaspases cleave proteins into peptides that comprise a P4-P3-P2-D amino acid sequence at the C-terminus, wherein P4 is not D.

An “anti-cleaved iCaspase substrate antibody” as used herein is an antibody that binds to a peptide produced by iCaspase cleavage. Such peptides comprises a P4-P3-P2-D amino acid sequence the C-terminus, wherein P4 is not D.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “chimeric” antibody refers to an antibody in which a portion of the heavy and/or light chain is derived from a particular source or species, while the remainder of the heavy and/or light chain is derived from a different source or species.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “immunoconjugate” is an antibody conjugated to one or more heterologous molecule(s), including but not limited to a cytotoxic agent.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the singular form “a” “an”, and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

### II. Compositions and Methods

A. Antibodies that Bind to a Cleaved iCaspase Substrate

1. Inflammatory Caspases

In one aspect, the present disclosure provides antibodies that interact with or otherwise bind to a region, such as an epitope, of a cleaved substrate of an inflammatory caspase (iCaspase). Caspases are cysteine-aspartic proteases that are synthesized as inactive zymogens (or, “pro-caspases”) that are only activated following an appropriate stimulus. Caspase activation involves dimerization and often oligomerization of the pro-caspases, followed by cleavage into small and large subunits. The large and small caspase subunits then associate with each other to form an active caspase heterodimer. In the case of inflammatory caspases (also known as “iCaspases”, or “iCasps”), activation is stimulated by inflammasomes, which are cytosolic, multi-protein complexes that sense patterns of pathogenesis or metabolic changes (Lamkanfi M Nat Rev Immunol 2011 11(3):213-220; Martinon F, et al. J Mol Cell 2002 10(2):417-426; Rathinam V A & Fitzgerald K A Cell 2016 165(4):792-800). Humans express three inflammatory caspases (caspases-1, -4, and -5), and mice express two inflammatory caspases (caspases-1 and -11).

In some embodiments, human caspase-1 is variously referred to as iCasp 1, CASP1, interleukin-1 beta converting enzyme (ICE), P45, or IL1BC. The amino acid sequence of human caspase-1 precursor (i.e., pro-caspase) is set forth below as SEQ ID NO: 21.

MADKVLKEKRKLFIRSMGEGTINGLLDELLQTRVLNKEEMEKVKRENATV
MDKTRALIDSVIPKGAQACQICITYICEEDSYLAGTLGLSADQTSGNYLN
MQDSQGVLSSFPAPQAVQDNPAMPTSSGSEGNVKLCSLEEAQRIWKQKSA
EIYPIMDKSSRTRLALIICNEEFDSIPRRTGAEVDITGMTMLLQNLGYSV
DVKKNLTASDMTTELEAFAHRPEHKTSDSTFLVFMSHGIREGICGKKHSE
QVPDILQLNAIFNMLNTKNCPSLKDKPKVIIIQACRGDSPGVVWFKDSVG
VSGNLSLPTTEEFEDDAIKKAHIEKDFIAFCSSTPDNVSWRHPTMGSVFI
GRLIEHMQEYACSCDVEEIFRKVRFSFEQPDGRAQMPTTERVTLTRCFYL
FPGH

In some embodiments, human caspase-4 is variously referred to as iCasp 4, CASP4, TX, Mih1, ICH-2, Mih1/TX, ICEREL-II, or ICE(rel)II. The amino acid sequence of human caspase-4 precursor is set forth below as SEQ ID NO: 22.

MAEGNHRKKPLKVLESLGKDFLTGVLDNLVEQNVLNWKEEEKKKYYDAKT
EDKVRVMADSMQEKQRMAGQMLLQTFFNIDQISPNKKAHPNMEAGPPESG
ESTDALKLCPHEEFLRLCKERAEEIYPIKERNNRTRLALIICNTEFDHLP
PRNGADFDITGMKELLEGLDYSVDVEENLTARDMESALRAFATRPEHKSS
DSTFLVLMSHGILEGICGTVHDEKKPDVLLYDTIFQIFNNRNCLSLKDKP
KVIIVQACRGANRGELWVRDSPASLEVASSQSSENLEEDAVYKTHVEKDF
IAFCSSTPHNVSWRDSTMGSIFITQLITCFQKYSWCCHLEEVFRKVQQSF
ETPRAKAQMPTIERLSMTRYFYLFPGN

In some embodiments, human caspase-5 is variously referred to as iCasp5, ICH-3, ICEREL-III, or ICE(rel)III. The amino acid sequence of human caspase-5 precursor isoform a is set forth below as SEQ ID NO: 23.

MAEDSGKKKRRKNFEAMFKGILQSGLDNFVINHMLKNNVAGQTSIQTLVP
NTDQKSTSVKKDNHKKKTVKMLEYLGKDVLHGVFNYLAKHDVLTLKEEEK
KKYYDTKIEDKALILVDSLRKNRVAHQMFTQTLLNMDQKITSVKPLLQIE
AGPPESAESTNILKLCPREEFLRLCKKNHDEIYPIKKREDRRRLALIICN
TKFDHLPARNGAHYDIVGMKRLLQGLGYTVVDEKNLTARDMESVLRAFAA
RPEHKSSDSTFLVLMSHGILEGICGTAHKKKKPDVLLYDTIFQIFNNRNC
LSLKDKPKVIIVQACRGEKHGELWVRDSPASLALISSQSSENLEADSVCK
IHEEKDFIAFCSSTPHNVSWRDRTRGSIFITELITCFQKYSCCCHLMEIF
RKVQKSFEVPQAKAQMPTIERATLTRDFYLFPGN

The amino acid sequence of murine caspase-1 precursor is set forth below as SEQ ID NO: 24.

MADKILRAKRKQFINSVSIGTINGLLDELLEKRVLNQEEMDKIKLANITA
MDKARDLCDHVSKKGPQASQIFITYICNEDCYLAGILELQSAPSAETFVA
TEDSKGGHPSSSETKEEQNKEDGTFPGLTGTLKFCPLEKAQKLWKENPSE
IYPIMNTTTRTRLALIICNTEFQHLSPRVGAQVDLREMKLLLEDLGYTVK
VKENLTALEMVKEVKEFAACPEHKTSDSTFLVFMSHGIQEGICGTTYSNE
VSDILKVDTIFQMMNTLKCPSLKDKPKVIIIQACRGEKQGVVLLKDSVRD
SEEDFLTDAIFEDDGIKKAHIEKDFIAFCSSTPDNVSWRHPVRGSLFIES
LIKHMKEYAWSCDLEDIFRKVRESFEQPEFRLQMPTADRVTLTKRFYLFP
GH

The amino acid sequence of murine caspase-11 precursor is set forth below as SEQ ID NO: 25.

MAENKHPDKPLKVLEQLGKEVLTEYLEKLVQSNVLKLKEEDKQKFNNAER
SDKRWVFVDAMKKKHSKVGEMLLQTFFSVDPGSHHGEANLEMEEPEESLN
TLKLCSPEEFTRLCREKTQEIYPIKEANGRTRKALIICNTEFKHLSLRYG
ANFDIIGMKGLLEDLGYDVVVKEELTAEGMESEMKDFAALSEHQTSDSTF
LVLMSHGTLHGICGTMHSEKTPDVLQYDTIYQIFNNCHCPGLRDKPKVII
VQACRGGNSGEMWIRESSKPQLCRGVDLPRNMEADAVKLSHVEKDFIAFY
STTPHHLSYRDKTGGSYFITRLISCFRKHACSCHLFDIFLKVQQSFEKAS
IHSQMPTIDRATLTRYFYLFPGN

Multiple different types of inflammasome complexes exist, which have been broadly classified as canonical or non-canonical inflammasomes. Upon detection of various microbial or endogenous stimuli by sensor proteins, canonical inflammasomes assemble and activate caspase-1, leading to cell death and production of pro-inflammatory cytokines (e.g., IL-1β and IL-18). More recently, the non-canonical inflammasome was discovered. The non-canonical inflammasome has distinct features from the canonical inflammasome. In non-canonical inflammasome activation, intracellular lipopolysaccharide (LPS) or oxidized phospholipids directly bind caspase-4/5/11, leading to caspase oligomerization, caspase activation, and inflammatory cell death or pyroptosis (see Zanoni I, et al. Science 2016 352(6290):1232-1236; Kayagaki N, et al. Nature 2011 479(7371):117-121; Shi J, et al. Nature 2014 514(7521):187-192; and Kayagaki N, et al. Science 2013 341(6151):1246-1249).

2. Canonical iCaspase Substrates

Antibodies that binds to a cleaved inflammatory caspase (iCaspase) substrate are provided herein. iCaspases and their substrates are involved in regulating cellular processes including pyroptosis, a highly inflammatory form of programmed cell death that can occur upon infection with intracellular pathogens. Specifically, a landmark in the inflammasome field was the discovery that pyroptosis is driven by iCaspase-mediated cleavage of gasdermin D (GSDMD) (Kayagaki N, et al. Nature 2015 526(7575):666-671; Shi J, et al. Nature 2015 526(7575):660-665). Cleavage of GSDMD relieves an auto-inhibited state, leading to assembly of a multimeric GSDMD pore in the plasma membrane, and release of cytoplasmic molecules (Ding J, et al. Nature 2016 535(7610):111-116; Liu X, et al. Nature 2016 535(7610):153-158).

While the initial discovery of the non-canonical inflammasome occurred in macrophages, many non-myeloid cells, such as endothelial and epithelial cells, express both caspase-11 and GSDMD, indicating that this essential pathway may have additional functions. For example, caspase-11 has been shown to control cytoplasmic bacterial growth independent of pyroptosis (Thurston T L, et al. Nat Commun 2016 7:13292). In another instance, caspase-11 confers protection in a model of inflammatory bowel disease due to activity in both the myeloid and non-myeloid compartments (Oficjalska K, et al. J Immunol 2015 194(3):1252-1260; Demon D, et al. Mucosal Immunol 2014 7(6):1480-1491).

Accordingly, in some embodiments, the iCaspase substrate is GSDMD. In some embodiments, the iCaspase substrate is human GSDMD (hGsdmd). In some embodiments, the iCaspase substrate is mouse GSDMD (mGsdmd). In some embodiments, the iCaspase substrate is caspase-11. Additional iCaspase substrates are known in the art. In some embodiments, the iCaspase substrate is IL1β. In some embodiments, the iCaspase substrate is human IL1β (hIL1β). In some embodiments, the iCaspase substrate is mouse IL1β (mIL1β). In some embodiments, the iCaspase substrate is IL18. In some embodiments, the iCaspase substrate is the canonical cleavage product of IL1β (B). In some embodiments, the iCaspase substrate is the noncanonical cleavage product of IL1β (A).

In some embodiments, an iCaspase substrate is cleaved by an iCaspase, thereby becoming a cleaved iCaspase substrate.

Notably, not all caspases are inflammatory caspases and, in particular, some caspases function in apoptosis instead. Such apoptotic caspases are also known as initiator and executioner caspases, depending on their role in apoptosis, and include caspase-2, caspase-3, caspase-6, caspase-7, and others. As described below, inflammatory and apoptotic caspases have different sequence-specificities, and therefore cleave target substrates at different protein sequence motifs.

Caspases in general selectively cleave substrates at primary sequence motifs with four positions (from N- to C-terminus: P4-P3-P2-P1) that contain an aspartic acid residue (also known as “Asp” or “D”) at P1, where P1 becomes the C-terminus of the cleaved protein fragment. Caspases in general tolerate sequence diversity at positions P2 and P3. At position 4 (P4), the apoptotic caspases (e.g., caspase-3, -6, and -7) prefer substrates with an aspartic acid (e.g., D-X-X-D). In contrast, the iCaspases prefer substrates with a hydrophobic P4 residue (e.g., W/I-X-X-D) (see Thornberry N A, et al. J Biol Chem 1997 272(29):17907-17911; Kang S J, et al. J Cell Biol 2000 149(3):613-622).

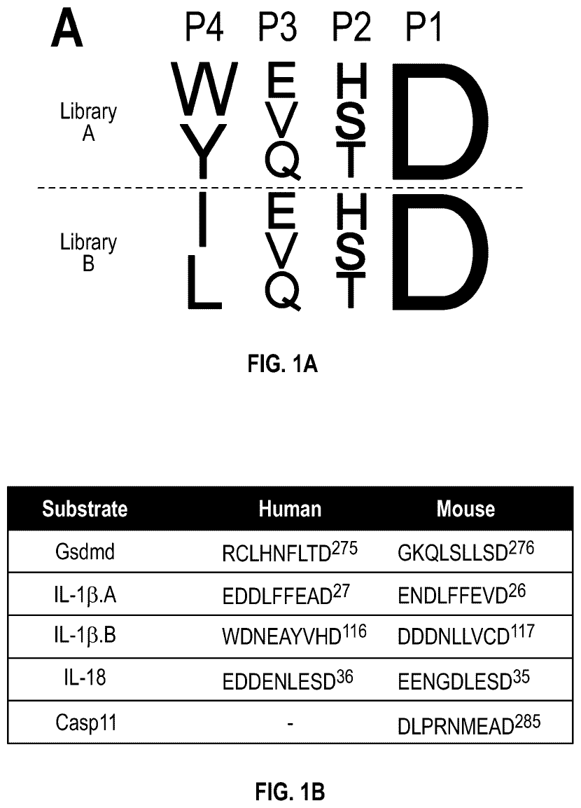

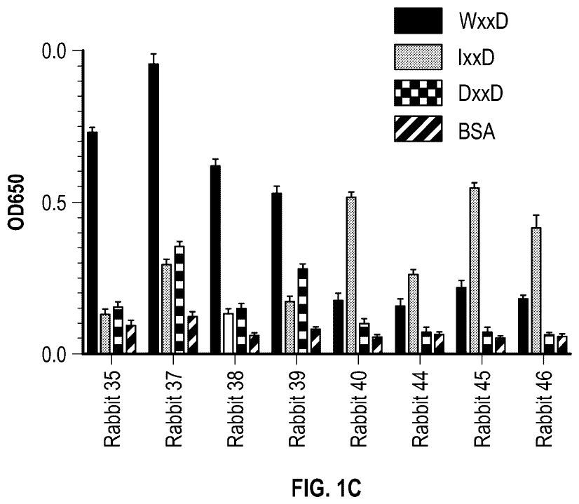

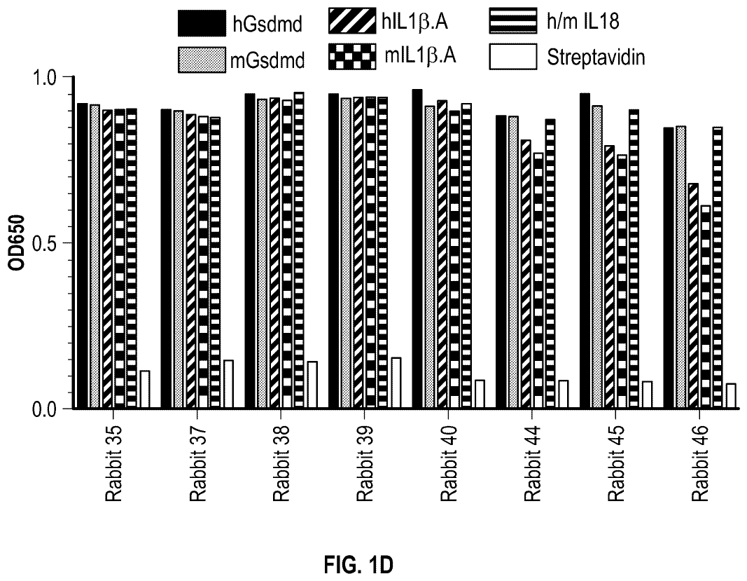

The present disclosure is based on the generation of antibodies that bind to cleaved iCaspase substrates. As described in detail herein, target and decoy peptide libraries were designed using recognition motifs for the iCasps and apoptotic caspases. Specifically, to select for antibodies that bind to cleaved iCaspase substrates, two peptide libraries were synthesized for use as target antigens, each shown from N- to C-terminus: W/Y-X-X-D and I/L-X-X-D, where P3 was an equimolar mixture of E, V, and Q and P2 was an equimolar mixture of H, S, and T (see FIG. 1A). As controls, two peptide libraries were synthesized in which P2 and P3 had the same degeneracy, but either the C-terminal carboxylate was capped with an amide (W-X-X-D-NH₂ or I-X-X-D-NH₂) or the P4 position was changed to D (D-X-X-D) (FIG. 1C). The D-X-X-D control libraries corresponds to the recognition motif of the apoptotic caspases, as noted above.

3. Antibodies that Bind to a Cleaved iCaspase Substrate

Antibodies that binds to a cleaved iCaspase substrate are provided herein. In some embodiments, the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid. In some embodiments, P4 is a hydrophobic amino acid. In some embodiments, P4 is selected from the group consisting of W, F, L, I, P, Y, V, and M. In some embodiments, P4 is W, I, L, or Y. In some embodiments, P4 is W or I. In some embodiments, P4 is W. In some embodiments, P4 is F. In some embodiments, P4 is I. In some embodiments, P4 is P. In some embodiments, P4 is Y. In some embodiments, P4 is V. In some embodiments, P4 is M. In some embodiments, P3 is E, V, Q, A, I, or L. In some embodiments, P3 is W. In some embodiments, P3 is F. In some embodiments, P3 is Y. In some embodiments, P3 is E. In some embodiments, P3 is V. In some embodiments, P3 is Q. In some embodiments, P3 is A. In some embodiments, P3 is I. In some embodiments, P3 is L. In some embodiments, P2 is H, S, or T. In some embodiments, P2 is H. In some embodiments, P2 is S. In some embodiments, P2 is T.

In some embodiments, an antibody that binds to a cleaved iCaspase substrate is provided, wherein the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid. In some embodiments, the antibody specifically binds to a peptide comprising the amino acid sequence P4-P3-P2-D at the C-terminus of the peptide as determined by an enzyme-linked immunosorbent assay (ELISA). In some embodiments, the ELISA measuring the ability of the antibody to specifically binds to a peptide comprising the amino acid sequence P4-P3-P2-D at the C-terminus of the peptide produces a signal. In some embodiments, the ELISA is performed with negative control sample included, for example, to produce a negative control sample signal. In some embodiments, the negative control sample is binding of the antibody to BSA. In some embodiments, the negative control sample is binding of the antibody to streptavidin. In some embodiments, binding of the antibody to a peptide comprising the amino acid sequence P4-P3-P2-D at the C-terminus of the peptide produces a signal that is more than 2, 3, 4, 5, 6, 7, 8, 9, 10, 20, 50, or 100-fold greater than a negative control sample signal. In some embodiments, binding of the antibody to a peptide comprising the amino acid sequence P4-P3-P2-D at the C-terminus of the peptide produces a signal that is statistically significantly greater than a negative control sample signal.

In some embodiments, the antibody binds to a peptide comprising the amino acid sequence P4-P3-P2-D at the C-terminus of the peptide with a greater affinity than a control antibody binds to the peptide. In some embodiments, the control antibody is an isotype control. In some embodiments, the antibody specifically binds to a peptide comprising the amino acid sequence P4-P3-P2-D at the C-terminus of the peptide in a Western blot. In some embodiments, the antibody can immunoprecipitate a peptide comprising the amino acid sequence P4-P3-P2-D at the C-terminus of the peptide. In some embodiments, the antibody can be co-crystallized with a peptide comprising the amino acid sequence P4-P3-P2-D at the C-terminus of the peptide. In some embodiments, the antibody binds a peptide comprising the amino acid sequence P4-P3-P2-D at the C-terminus of the peptide in a surface plasmon resonance (SPR) assay.

In some embodiments, an antibody that binds to a cleaved iCaspase substrate is provided, wherein the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid. In some embodiments, the antibody binds to a peptide comprising the amino acid sequence P4-P3-P2-D at the C-terminus of the peptide with a dissociation constant (K_{d}) that is less than 100, 10, 1, or 0.1 μM. In some embodiments, the antibody binds to a peptide comprising the amino acid sequence P4-P3-P2-D at the C-terminus of the peptide with a dissociation constant (K_{d}) that is about 0.1, 0.2, 0.3, 0.4, 0.5, 0.6, 0.7, 0.8, 0.9, 1, 2, 3, 4, 5, 6, 7, 8, 9, 10, 15, 20, 25, 50, 75, or 100 μM including any value or range between these values. In some embodiments, the antibody binds to a peptide comprising the amino acid sequence P4-P3-P2-D at the C-terminus of the peptide with a K_{d} that is less than 100, 10, or 1 nM. In some embodiments, the antibody binds to a peptide comprising the amino acid sequence P4-P3-P2-D at the C-terminus of the peptide with a dissociation constant (K_{d}) that is about 5, 10, 20, 30, 40, 50, 60, 70, 80, 90, 100, 250, 500, 750, or 1000 nM, including any value or range between these values. In some embodiments, the antibody binds to a peptide comprising the amino acid sequence P4-P3-P2-D at the C-terminus of the peptide with a K_{d} that is less than 100 μM. In some embodiments, the antibody binds to a peptide comprising the amino acid sequence P4-P3-P2-D at the C-terminus of the peptide with a K_{d} of 1-10 μM.

In some embodiments, the antibody does not bind to peptides comprising the amino acid sequence D-X-X-D or E-X-X-D at the C-terminus, wherein X is any amino acid. In some embodiments, the antibody does not bind to peptides comprising the amino acid sequence D-X-X-D or E-X-X-D at the C-terminus, wherein binding of the antibody is not detectable over background or is at the same level as a negative control. In some embodiments, background is the level of non-specific binding. In some embodiments, background is the level of binding of the antibody to streptavidin. In some embodiments, background is the level of binding of the antibody to bovine serum albumin (BSA). In some embodiments, binding of the antibody to peptides comprising the amino acid sequence D-X-X-D or E-X-X-D at the C-terminus is not detectable. In some embodiments, binding of the antibody to peptides comprising the amino acid sequence D-X-X-D or E-X-X-D at the C-terminus is not detectable by immunoprecipitation. In some embodiments, binding of the antibody to peptides comprising the amino acid sequence D-X-X-D or E-X-X-D at the C-terminus is not detectable in a Western blot. In some embodiments, binding of the antibody to peptides comprising the amino acid sequence D-X-X-D or E-X-X-D at the C-terminus is not detectable in an ELISA. In some embodiments, the antibody binds to peptides comprising the amino acid sequence D-X-X-D or E-X-X-D at the C-terminus to that same extent that the antibody binds BSA. In some embodiments, the antibody binds to peptides comprising the amino acid sequence D-X-X-D or E-X-X-D at the C-terminus to that same extent that the antibody binds streptavidin. In some embodiments, binding of the antibody to peptides comprising the amino acid sequence D-X-X-D or E-X-X-D at the C-terminus is assessed in an ELISA in which as negative control sample is included, for example, to produce a negative control sample signal. In some embodiments, the negative control sample is binding of the antibody to BSA. In some embodiments, the negative control sample is binding of the antibody to streptavidin. In some embodiments, the ELISA assessing the ability of the antibody to bind peptides comprising the amino acid sequence D-X-X-D or E-X-X-D at the C-terminus produces a signal. In some embodiments, the signal is the same as a negative control sample signal. In some embodiments, the signal is not statistically significantly different from a negative control sample signal. In some embodiments, the signal is no more than 1.1, 1.2, 1.3, 1.4, or 1.5-fold above or below the negative control sample signal.

In some embodiments, the antibody that binds to a cleaved iCaspase substrate comprises one, two, three, four, five, or six CDRs of antibody CJ11 as shown in Table 2. In some embodiments, the antibody comprises the VH and/or the VL of antibody CJ11 as shown in Table 1. In some embodiments, the antibody comprises the heavy chain and/or the light chain of antibody CJ11 as shown in Table 3. In some embodiments, the antibody comprises the heavy chain of antibody CJ11 comprising an amino acid substitution, as shown in Table 5. In some embodiments, the antibody comprises a T99R substitution in the heavy chain. In some embodiments, the antibody comprises a Y32R substitution in the heavy chain.

In some embodiments, the antibody that binds to a cleaved iCaspase substrate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In certain embodiments, a VH sequence contains substitutions (e.g., conservative substitutions), insertions, or deletions relative to the amino acid sequence of SEQ ID NO:1, but retains the ability to bind a cleaved iCaspase substrate as the antibody comprising SEQ ID NO:1. In certain embodiments, a total of 1 to 13 amino acids have been substituted, inserted and/or deleted in SEQ ID NO: 1. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO:3, (b) a CDR-H2 comprising the amino acid sequence of SEQ ID NO:4, and (c) a CDR-H3 comprising the amino acid sequence of SEQ ID NO:5.

In another aspect, an antibody that binds to a cleaved iCaspase substrate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, a VL sequence contains substitutions (e.g., conservative substitutions), insertions, or deletions relative to the amino acid sequence of SEQ ID NO:2, but retains the ability to bind a cleaved iCaspase substrate as the antibody comprising SEQ ID NO:2. In certain embodiments, a total of 1 to 11 amino acids have been substituted, inserted and/or deleted in SEQ ID NO:2. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO:6; (b) a CDR-L2 comprising the amino acid sequence of SEQ ID NO:7; and (c) a CDR-L3 comprising the amino acid sequence of SEQ ID NO:8.

In one embodiment, the antibody that binds to a cleaved iCaspase substrate comprises a VL comprising the amino acid sequence of SEQ ID NO:2 and a VH comprising the amino acid sequence of SEQ ID NO:1.

In another aspect, an antibody that binds to a cleaved iCaspase substrate is provided, wherein the antibody comprises a VH comprising a CDR-H1 comprising the amino acid sequence of SEQ ID NO:3, a CDR-H2 comprising the amino acid sequence of SEQ ID NO:3, and a CDR-H3 comprising the amino acid sequence of SEQ ID NO:5; and a VL comprising a CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8.

In another aspect, an antibody that binds to a cleaved iCaspase substrate is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:1; and a VL CDR1, a VL CDR2, and a VL CDR3, respectively comprising the amino acid sequences of a CDR1, a CDR2, and a CDR3 of a VL having the sequence set forth in SEQ ID NO:2.

In some embodiments, the antibody that binds to a cleaved iCaspase substrate comprises a heavy chain comprising the amino acid sequence of SEQ ID NO:17 and a light chain comprising the amino acid sequence of SEQ ID NO:18. In some embodiments, the antibody that binds to a cleaved iCaspase substrate comprises a heavy chain comprising the amino acid sequence of SEQ ID NO:17 without the C-terminal lysine residue, and a light chain comprising the amino acid sequence of SEQ ID NO:18. In some embodiments, the antibody comprises a T99R substitution in the heavy chain. In some embodiments, the antibody comprises a Y32R substitution in the heavy chain. In some embodiments, the antibody that binds to a cleaved iCaspase substrate comprises a heavy chain comprising the amino acid sequence of SEQ ID NO:21 and a light chain comprising the amino acid sequence of SEQ ID NO:18. In some embodiments, the antibody that binds to a cleaved iCaspase substrate comprises a heavy chain comprising the amino acid sequence of SEQ ID NO:21 without the C-terminal lysine residue, and a light chain comprising the amino acid sequence of SEQ ID NO:18. In some embodiments, the antibody that binds to a cleaved iCaspase substrate comprises a heavy chain comprising the amino acid sequence of SEQ ID NO:22 and a light chain comprising the amino acid sequence of SEQ ID NO:18. In some embodiments, the antibody that binds to a cleaved iCaspase substrate comprises a heavy chain comprising the amino acid sequence of SEQ ID NO:22 without the C-terminal lysine residue, and a light chain comprising the amino acid sequence of SEQ ID NO:18. In some embodiments, the antibody that binds to a cleaved iCaspase substrate comprises a heavy chain comprising the amino acid sequence of SEQ ID NO:23 and a light chain comprising the amino acid sequence of SEQ ID NO:18. In some embodiments, the antibody that binds to a cleaved iCaspase substrate comprises a heavy chain comprising the amino acid sequence of SEQ ID NO:23 without the C-terminal lysine residue, and a light chain comprising the amino acid sequence of SEQ ID NO:18.

In some embodiments, the antibody that binds to a cleaved iCaspase substrate comprises one, two, three, four, five, or six CDRs of antibody CJ2 as shown in Table 2. In some embodiments, the antibody comprises the VH and/or the VL of antibody CJ2 as shown in Table 1. In some embodiments, the antibody comprises the heavy chain and/or the light chain of antibody CJ2 as shown in Table 3. In some embodiments, the antibody comprises the heavy chain of antibody CJ2 comprising an amino acid substitution, as shown in Table 5. In some embodiments, the antibody comprises a T99R substitution in the heavy chain. In some embodiments, the antibody comprises a Y32R substitution in the heavy chain.

In some embodiments, the antibody that binds to a cleaved iCaspase substrate comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:9. In certain embodiments, a VH sequence contains substitutions (e.g., conservative substitutions), insertions, or deletions relative to the amino acid sequence of SEQ ID NO:9, but retains the ability to bind a cleaved iCaspase substrate as the antibody comprising SEQ ID NO:9. In certain embodiments, a total of 1 to 13 amino acids have been substituted, inserted and/or deleted in SEQ ID NO: 9. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO:11, (b) a CDR-H2 comprising the amino acid sequence of SEQ ID NO:12, and (c) a CDR-H3 comprising the amino acid sequence of SEQ ID NO:13.

In another aspect, an antibody that binds to a cleaved iCaspase substrate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:10. In certain embodiments, a VL sequence contains substitutions (e.g., conservative substitutions), insertions, or deletions relative to the amino acid sequence of SEQ ID NO:10, but retains the ability to bind a cleaved iCaspase substrate as the antibody comprising SEQ ID NO:10. In certain embodiments, a total of 1 to 11 amino acids have been substituted, inserted and/or deleted in SEQ ID NO:10. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO:14; (b) a CDR-L2 comprising the amino acid sequence of SEQ ID NO:15; and (c) a CDR-L3 comprising the amino acid sequence of SEQ ID NO:16.

In one embodiment, the antibody that binds to a cleaved iCaspase substrate comprises a VL comprising the amino acid sequence of SEQ ID NO:10 and a VH comprising the amino acid sequence of SEQ ID NO:9.

In another aspect, an antibody that binds to a cleaved iCaspase substrate is provided, wherein the antibody comprises a VH comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13; and a VL comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16.

In another aspect, an antibody that binds to a cleaved iCaspase substrate is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:9; and a VL CDR1, a VL CDR2, and a VL CDR3, respectively comprising the amino acid sequences of a CDR1, a CDR2, and a CDR3 of a VL having the sequence set forth in SEQ ID NO:10.

In some embodiments, the antibody that binds to a cleaved iCaspase substrate comprises a heavy chain comprising the amino acid sequence of SEQ ID NO:19 and a light chain comprising the amino acid sequence of SEQ ID NO:20. In some embodiments, the antibody that binds to a cleaved iCaspase substrate comprises a heavy chain comprising the amino acid sequence of SEQ ID NO:19 without the C-terminal lysine residue, and a light chain comprising the amino acid sequence of SEQ ID NO:20. In some embodiments, the antibody comprises a T99R substitution in the heavy chain. In some embodiments, the antibody comprises a Y32R substitution in the heavy chain. In some embodiments, the antibody that binds to a cleaved iCaspase substrate comprises a heavy chain comprising the amino acid sequence of SEQ ID NO:24 and a light chain comprising the amino acid sequence of SEQ ID NO:20. In some embodiments, the antibody that binds to a cleaved iCaspase substrate comprises a heavy chain comprising the amino acid sequence of SEQ ID NO:24 without the C-terminal lysine residue, and a light chain comprising the amino acid sequence of SEQ ID NO:20. In some embodiments, the antibody that binds to a cleaved iCaspase substrate comprises a heavy chain comprising the amino acid sequence of SEQ ID NO:25 and a light chain comprising the amino acid sequence of SEQ ID NO:20. In some embodiments, the antibody that binds to a cleaved iCaspase substrate comprises a heavy chain comprising the amino acid sequence of SEQ ID NO:25 without the C-terminal lysine residue, and a light chain comprising the amino acid sequence of SEQ ID NO:20. In some embodiments, the antibody that binds to a cleaved iCaspase substrate comprises a heavy chain comprising the amino acid sequence of SEQ ID NO:26 and a light chain comprising the amino acid sequence of SEQ ID NO:20. In some embodiments, the antibody that binds to a cleaved iCaspase substrate comprises a heavy chain comprising the amino acid sequence of SEQ ID NO:26 without the C-terminal lysine residue, and a light chain comprising the amino acid sequence of SEQ ID NO:20.

In some embodiments, the antibody that binds to a cleaved iCaspase substrate comprises an amino acid substitution. In some embodiments, the antibody comprises an amino acid substitution that improves binding to a cleaved iCaspase substrate relative to the parental antibody. In some embodiments, the antibody specifically binds to a peptide comprising the amino acid sequence P4-P3-P2-D at the C-terminus of the peptide. In some embodiments, the antibody comprises an amino acid substitution that improves binding to the amino acid sequence P4-P3-P2-D at the C-terminus of the peptide relative to the parental antibody. In some embodiments, the antibody comprises an amino acid substitution that enhances recognition of the D residue of the amino acid sequence P4-P3-P2-D at the C-terminus of the peptide. In some embodiments, the antibody comprises an amino acid substitution in the heavy chain of the antibody. In some embodiments, the antibody comprises a T99R substitution in the heavy chain. In some embodiments, the antibody comprises a Y32R substitution in the heavy chain. In some embodiments, the antibody comprises a T99R or Y32R substitution, and binds with enhanced recognition of the D residue of the amino acid sequence P4-P3-P2-D at the C-terminus of the peptide.

In another aspect, an antibody that binds to a cleaved iCaspase substrate is provided, wherein the antibody comprises a VH as in any of the embodiments provided above, and a VL as in any of the embodiments provided above.

In a further aspect of the invention, an antibody that binds to a cleaved iCaspase substrate according to any of the above embodiments is a monoclonal antibody, including a chimeric, humanized or human antibody. In some embodiments, the antibody that binds to a cleaved iCaspase substrate is an antibody fragment, e.g., a Fv, Fab, Fab′, scFv, diabody, or F(ab′)₂ fragment. In another embodiment, the antibody that binds to a cleaved iCaspase substrate is a full-length antibody, e.g., an intact IgG1 antibody or other antibody class or isotype as defined herein. In some embodiments, the antibody is a full-length antibody, a Fab fragment, or an scFv. In some embodiments, the antibody is of the IgA, IgD, IgE, IgG, or IgM class. In some embodiments, the antibody is of the IgG class. In some embodiments, the antibody is of the IgG class and has an IgG₁, IgG₂, IgG₃, or IgG₄ isotype. In some embodiments, the antibody is of the IgA class and has an IgA₁ or IgA₂ isotype.

In some embodiments, the antibody is a rabbit antibody, rodent antibody, or goat antibody. In some embodiments, the antibody is a rabbit antibody that possesses an amino acid sequence which corresponds to that of an antibody produced by a rabbit or a rabbit cell or derived from a non-rabbit source that utilizes rabbit antibody repertoires or other rabbit antibody-encoding sequences. In some embodiments, the antibody is derived from a rabbit. In some embodiments, the antibody is derived from a New Zealand White Rabbit. In some embodiments, the antibody is derived from a rodent. In some embodiments, the antibody is derived from a goat. In some embodiments, the antibody comprises a Fc region derived from a rabbit, goat, or rodent antibody. In some embodiments, the antibody comprises an antibody fragment from a rabbit, goat, or rodent antibody.

In a further aspect of the invention, an antibody that binds to a cleaved iCaspase substrate according to any of the above embodiments or described herein is conjugated to a heterologous moiety, agent, or label. Examples of suitable labels are those numerous labels known for use in immunoassay, including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵, ³H, and ¹³¹I, fluorophores such as rare-earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, HRP, alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-p-galactosidase with MUG), spin labels, bacteriophage labels, stable free radicals, and the like. In some embodiments, the label is selected from the group consisting of biotin, digoxigenin, and fluorescein. In some embodiments, the antibody that binds to a cleaved iCaspase substrate according to any of the above embodiments is conjugated to biotin.

In another aspect, provided herein is a composition comprising one or more of the antibodies that bind to a cleaved iCaspase substrate according to any of the above embodiments or described herein. Also provided herein is a nucleic acid encoding an antibody that binds to a cleaved iCaspase substrate described herein, a vector comprising the nucleic acid, and a host cell comprising the vector, as described in further detail below. In some embodiments, the host cell is isolated or purified. In some embodiments, the host cell is a cell culture medium.

B. Host Cells, Nucleic Acid

Also provided herein is nucleic acid encoding an antibody that binds to a cleaved iCaspase substrate. In some embodiments, the nucleic acid encodes any of the antibodies described herein.

In some embodiments, the nucleic acid encodes an antibody that binds to a cleaved iCaspase substrate comprising one, two, three, four, five, or six CDRs of antibody CJ11 as shown in Table 2. In some embodiments, the nucleic acid encodes an antibody comprising the VH and/or the VL of antibody CJ11 as shown in Table 1. In some embodiments, the nucleic acid encodes an antibody comprising the heavy chain and/or the light chain of antibody CJ11 as shown in Table 3. In some embodiments, the antibody comprises the heavy chain of antibody CJ11 comprising an amino acid substitution, as shown in Table 5. In some embodiments, the antibody comprises a T99R substitution in the heavy chain. In some embodiments, the antibody comprises a Y32R substitution in the heavy chain.

In some embodiments, the nucleic acid encodes an antibody that binds to a cleaved iCaspase substrate comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In certain embodiments, the nucleic acid encodes a VH sequence that contains substitutions (e.g., conservative substitutions), insertions, or deletions relative to the amino acid sequence of SEQ ID NO:1, but retains the ability to bind a cleaved iCaspase substrate as the antibody comprising SEQ ID NO:1. In certain embodiments, a total of 1 to 13 amino acids have been substituted, inserted and/or deleted in SEQ ID NO: 1. In certain embodiments, substitutions, insertions, or deletions occur in regions outside the CDRs (i.e., in the FRs). In a particular embodiment, the nucleic acid encodes a VH that comprises one, two or three CDRs selected from the group consisting of: (a) a CDR-H1 comprising the amino acid sequence of SEQ ID NO:3, (b) a CDR-H2 comprising the amino acid sequence of SEQ ID NO:4, and (c) a CDR-H3 comprising the amino acid sequence of SEQ ID NO:5.

In another aspect, a nucleic acid that encodes an antibody that binds to a cleaved iCaspase substrate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, the nucleic acid encodes a VL sequence that contains substitutions (e.g., conservative substitutions), insertions, or deletions relative to the amino acid sequence of SEQ ID NO:2, but retains the ability to bind a cleaved iCaspase substrate as the antibody comprising SEQ ID NO:2. In certain embodiments, a total of 1 to 11 amino acids have been substituted, inserted and/or deleted in SEQ ID NO:2. In certain embodiments, the substitutions, insertions, or deletions occur in regions outside the CDRs (i.e., in the FRs). In a particular embodiment, the nucleic acid encodes a VL that comprises one, two or three CDRs selected from the group consisting of (a) a CDR-L1 comprising the amino acid sequence of SEQ ID NO:6; (b) a CDR-L2 comprising the amino acid sequence of SEQ ID NO:7; and (c) a CDR-L3 comprising the amino acid sequence of SEQ ID NO:8.

In one embodiment, the nucleic acid encodes an antibody that binds to a cleaved iCaspase substrate comprising a VL comprising the amino acid sequence of SEQ ID NO:2 and a VH comprising the amino acid sequence of SEQ ID NO:1.

In another aspect, a nucleic acid that encodes an antibody that binds to a cleaved iCaspase substrate is provided, wherein the antibody comprises a VH comprising a CDR-H1 comprising the amino acid sequence of SEQ ID NO:3, a CDR-H2 comprising the amino acid sequence of SEQ ID NO:3, and a CDR-H3 comprising the amino acid sequence of SEQ ID NO:5; and a VL comprising a CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8.

In another aspect, a nucleic acid that encodes an antibody that binds to a cleaved iCaspase substrate is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:1; and a VL CDR1, a VL CDR2, and a VL CDR3, respectively comprising the amino acid sequences of a CDR1, a CDR2, and a CDR3 of a VL having the sequence set forth in SEQ ID NO:2.

In one embodiment, the nucleic acid encodes an antibody that binds to a cleaved iCaspase substrate comprising a heavy chain comprising the amino acid sequence of SEQ ID NO:17 and a light chain comprising the amino acid sequence of SEQ ID NO:18. In some embodiments, the antibody comprises a T99R substitution in the heavy chain. In some embodiments, the antibody comprises a Y32R substitution in the heavy chain. In some embodiments, the antibody that binds to a cleaved iCaspase substrate comprises a heavy chain comprising the amino acid sequence of SEQ ID NO:21 and a light chain comprising the amino acid sequence of SEQ ID NO:18. In some embodiments, the antibody that binds to a cleaved iCaspase substrate comprises a heavy chain comprising the amino acid sequence of SEQ ID NO:21 without the C-terminal lysine residue, and a light chain comprising the amino acid sequence of SEQ ID NO:18. In some embodiments, the antibody that binds to a cleaved iCaspase substrate comprises a heavy chain comprising the amino acid sequence of SEQ ID NO:22 and a light chain comprising the amino acid sequence of SEQ ID NO:18. In some embodiments, the antibody that binds to a cleaved iCaspase substrate comprises a heavy chain comprising the amino acid sequence of SEQ ID NO:22 without the C-terminal lysine residue, and a light chain comprising the amino acid sequence of SEQ ID NO:18. In some embodiments, the antibody that binds to a cleaved iCaspase substrate comprises a heavy chain comprising the amino acid sequence of SEQ ID NO:23 and a light chain comprising the amino acid sequence of SEQ ID NO:18. In some embodiments, the antibody that binds to a cleaved iCaspase substrate comprises a heavy chain comprising the amino acid sequence of SEQ ID NO:23 without the C-terminal lysine residue, and a light chain comprising the amino acid sequence of SEQ ID NO:18.

In some embodiments, the nucleic acid encodes an antibody that binds to a cleaved iCaspase substrate comprising one, two, three, four, five, or six CDRs of antibody CJ2 as shown in Table 2. In some embodiments, the nucleic acid encodes an antibody comprising the VH and/or the VL of antibody CJ2 as shown in Table 1. In some embodiments, the nucleic acid encodes an antibody comprising the heavy chain and/or the light chain of antibody CJ2 as shown in Table 3. In some embodiments, the antibody comprises the heavy chain of antibody CJ2 comprising an amino acid substitution, as shown in Table 5. In some embodiments, the antibody comprises a T99R substitution in the heavy chain. In some embodiments, the antibody comprises a Y32R substitution in the heavy chain.

In some embodiments, the nucleic acid encodes an antibody that binds to a cleaved iCaspase substrate comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO:9. In certain embodiments, the nucleic acid encodes a VH sequence contains substitutions (e.g., conservative substitutions), insertions, or deletions relative to the amino acid sequence of SEQ ID NO:9, but retains the ability to bind a cleaved iCaspase substrate as the antibody comprising SEQ ID NO:9. In certain embodiments, a total of 1 to 13 amino acids have been substituted, inserted and/or deleted in SEQ ID NO: 9. In certain embodiments, substitutions, insertions, or deletions occur in regions outside the CDRs (i.e., in the FRs). In a particular embodiment, the nucleic acid encodes a VH that comprises one, two or three CDRs selected from the group consisting of: (a) a CDR-H1 comprising the amino acid sequence of SEQ ID NO:11, (b) a CDR-H2 comprising the amino acid sequence of SEQ ID NO:12, and (c) a CDR-H3 comprising the amino acid sequence of SEQ ID NO:13.

In another aspect, a nucleic acid that encodes an antibody that binds to a cleaved iCaspase substrate is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:10. In certain embodiments, the nucleic acid encodes a VL sequence that contains substitutions (e.g., conservative substitutions), insertions, or deletions relative to the amino acid sequence of SEQ ID NO:10, but retains the ability to bind a cleaved iCaspase substrate as the antibody comprising SEQ ID NO:10. In certain embodiments, a total of 1 to 11 amino acids have been substituted, inserted and/or deleted in SEQ ID NO:10. In certain embodiments, the substitutions, insertions, or deletions occur in regions outside the CDRs (i.e., in the FRs). In a particular embodiment, the nucleic acid encodes a VL that comprises one, two or three CDRs selected from the group consisting of (a) a CDR-L1 comprising the amino acid sequence of SEQ ID NO:14; (b) a CDR-L2 comprising the amino acid sequence of SEQ ID NO:15; and (c) a CDR-L3 comprising the amino acid sequence of SEQ ID NO:16.

In one embodiment, the nucleic acid encodes an antibody that binds to a cleaved iCaspase substrate comprising a VL comprising the amino acid sequence of SEQ ID NO:10 and a VH comprising the amino acid sequence of SEQ ID NO:9.

In another aspect, a nucleic acid that encodes an antibody that binds to a cleaved iCaspase substrate is provided, wherein the antibody comprises a VH comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13; and a VL comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16.

In another aspect, a nucleic acid that encodes an antibody that binds to a cleaved iCaspase substrate is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:9; and a VL CDR1, a VL CDR2, and a VL CDR3, respectively comprising the amino acid sequences of a CDR1, a CDR2, and a CDR3 of a VL having the sequence set forth in SEQ ID NO:10.

In one embodiment, the nucleic acid encodes an antibody that binds to a cleaved iCaspase substrate comprising a heavy chain comprising the amino acid sequence of SEQ ID NO:19 and a light chain comprising the amino acid sequence of SEQ ID NO:20. In some embodiments, the antibody comprises a T99R substitution in the heavy chain. In some embodiments, the antibody comprises a Y32R substitution in the heavy chain. In some embodiments, the antibody that binds to a cleaved iCaspase substrate comprises a heavy chain comprising the amino acid sequence of SEQ ID NO:24 and a light chain comprising the amino acid sequence of SEQ ID NO:20. In some embodiments, the antibody that binds to a cleaved iCaspase substrate comprises a heavy chain comprising the amino acid sequence of SEQ ID NO:24 without the C-terminal lysine residue, and a light chain comprising the amino acid sequence of SEQ ID NO:20. In some embodiments, the antibody that binds to a cleaved iCaspase substrate comprises a heavy chain comprising the amino acid sequence of SEQ ID NO:25 and a light chain comprising the amino acid sequence of SEQ ID NO:20. In some embodiments, the antibody that binds to a cleaved iCaspase substrate comprises a heavy chain comprising the amino acid sequence of SEQ ID NO:25 without the C-terminal lysine residue, and a light chain comprising the amino acid sequence of SEQ ID NO:20. In some embodiments, the antibody that binds to a cleaved iCaspase substrate comprises a heavy chain comprising the amino acid sequence of SEQ ID NO:26 and a light chain comprising the amino acid sequence of SEQ ID NO:20. In some embodiments, the antibody that binds to a cleaved iCaspase substrate comprises a heavy chain comprising the amino acid sequence of SEQ ID NO:26 without the C-terminal lysine residue, and a light chain comprising the amino acid sequence of SEQ ID NO:20.

In some embodiments, the nucleic acid encodes an antibody that binds to a cleaved iCaspase substrate, wherein the antibody comprises an amino acid substitution. In some embodiments, the antibody comprises a T99R substitution in the heavy chain. In some embodiments, the antibody comprises a Y32R substitution in the heavy chain. In some embodiments, the antibody comprises a T99R or Y32R substitution, and binds with enhanced recognition of the D residue of the amino acid sequence P4-P3-P2-D at the C-terminus of the peptide.

In another aspect, a nucleic acid that encodes an antibody that binds to a cleaved iCaspase substrate is provided, wherein the antibody comprises a VH as in any of the embodiments provided above, and a VL as in any of the embodiments provided herein.

In a further aspect of the invention, the nucleic acid that encodes an antibody that binds to a cleaved iCaspase substrate according to any of the above embodiments encodes a monoclonal antibody, including a chimeric, humanized or human antibody. In some embodiments, the nucleic acid encodes a rabbit, rodent, or goat antibody. In some embodiments, the nucleic acid encodes an antibody that binds to a cleaved iCaspase substrate, wherein the antibody is an antibody fragment, e.g., a Fv, Fab, Fab′, scFv, diabody, or F(ab′)₂ fragment. In another embodiment, the nucleic acid encodes an antibody that binds to a cleaved iCaspase substrate wherein the antibody is a full-length antibody, e.g., an intact IgG1 antibody or other antibody class or isotype as defined herein. In some embodiments, the nucleic acid encodes an antibody that binds to a cleaved iCaspase substrate wherein the antibody is a full-length antibody, a Fab fragment, or an scFv fragment.

In some embodiments, the nucleic acid provided herein are in one or more vectors. For example, in some embodiments, provided herein is a vector comprising a heavy and light chain of an anti-cleaved iCaspase antibody. In some embodiments, the heavy and light chains are in different vectors.

For antibody production the humanized heavy and light chain expression vectors may be introduced into appropriate production cell lines know in the art such as, for example, NS0 cells. Introduction of the expression vectors may be accomplished by co-transfection via electroporation or any other suitable transformation technology available in the art. Antibody producing cell lines can then be selected and expanded and humanized antibodies purified. The purified antibodies can then be analyzed by standard techniques such as SDS-PAGE.

Also provided is a host cell comprising a nucleic acid encoding an antibody that binds to a cleaved iCaspase substrate. Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

C. Method of Screening

In some embodiments, provided herein is a method of screening for an antibody that binds to a cleaved iCaspase substrate, wherein the antibody specifically binds to a peptide comprising the amino acid sequence P4-P3-P2-D at the C-terminus of the peptide, wherein the antibody does not bind to peptides comprising the amino acid sequence D-X-X-D at the C-terminus, wherein X is any amino acid.

In some embodiments, the method comprising providing an antibody library and positively selecting antibodies that bind to a peptide comprising the amino acid sequence P4-P3-P2-D at the C-terminus of the peptide. In some embodiments, a phage display library is provided. In some embodiments, a yeast display library display library is provided. In some embodiments, a bacterial display library is provided.

The antibody libraries provided herein may comprises antibodies from various sources. For example in some embodiments, a library of synthetic antibodies is provided. In some embodiments, a library of human naïve antibodies is provided. In some embodiments, a library of camel antibodies is provided. In some embodiments, a murine antibody library is provided. In some embodiments, a library of rabbit antibodies is provided. In some embodiments, a library of humanized antibodies is provided.

In some embodiments, the library is produced by cloning antibodies from an immunized mammal. In some embodiments, the immunized mammal is a rodent or a rabbit. In some embodiments, the mammal is immunized with a peptide library. In some embodiments, the mammal is immunized with a library of cleaved iCaspase substrates. In some embodiments, the mammal is immunized with peptides comprising X-X-X-D at the C-terminus. In some embodiments, the mammal is immunized with peptides comprising P4-P3-P2-D at the C terminus, wherein P4 is a hydrophobic amino acid. In some embodiments, the mammal is immunized with peptides comprising P4-P3-P2-D at the C-terminus, wherein P4 is not D.

In some embodiments, the mammal is immunized with a library comprising peptides comprising W-X-X-D at the C terminus, wherein X is any amino acid. In some embodiments, the mammal is immunized with a library comprising peptides comprising Y-X-X-D at the C terminus, wherein X is any amino acid. In some embodiments, the mammal is immunized with peptides comprising I-X-X-D at the C-terminus, wherein X is any amino acid. In some embodiments, the mammal is immunized with peptides comprising L-X-X-D at the C-terminus, wherein X is any amino acid. In some embodiments, the mammal is immunized with a library of peptides comprising Y-X-X-D, I-X-X-D, L-X-X-D, and W-X-X-D at the C-terminus.

In some embodiments, the mammal is immunized with peptides comprising Y-P3-P2-D at the C-terminus, wherein P3 is an equimolar mixture of E, V, and Q and P2 is an equimolar mixture of H, S, and T. In some embodiments, the mammal is immunized with peptides comprising W-P3-P2-D at the C-terminus, wherein P3 is an equimolar mixture of E, V, and Q and P2 is an equimolar mixture of H, S, and T. In some embodiments, the mammal is immunized with peptides comprising I-P3-P2-D at the C-terminus, wherein P3 is an equimolar mixture of E, V, and Q and P2 is an equimolar mixture of H, S, and T. In some embodiments, the mammal is immunized with peptides comprising L-P3-P2-D at the C-terminus, wherein P3 is an equimolar mixture of E, V, and Q and P2 is an equimolar mixture of H, S, and T. In some embodiments, the mammal is immunized with a library of peptides comprising Y-P3-P2-D, W-P3-P2-D, I-P3-P2-D, and L-P3-P2-D at the C-terminus, wherein P3 is an equimolar mixture of E, V, and Q and P2 is an equimolar mixture of H, S, and T.

Also provided herein is a peptide library which can be used for producing and/or screening for antibodies that bind to a cleaved iCaspase substrate.

In some embodiments, the library comprises scFv antibodies. In some embodiments, the library comprises Fab fragments. In some embodiments, the library comprises full length antibodies.

In some embodiments, the antibody library is positively selected for antibodies that bind to a peptide comprising the amino acid sequence P4-P3-P2-D at the C-terminus. In some embodiments, the antibody library is positively selected by phage panning. In some embodiments, the antibody library is incubated with one or more peptides comprising the amino acid sequence P4-P3-P2-D motif at the C-terminus bound to a solid support. In some embodiments, the unbound antibodies are removed by washing and the bound antibodies are eluted with HCl. In some embodiments, the library is positively selected as least twice, at least three times, at least four times, or more than 5 times.

In some embodiments, the antibody library is positively selected by incubating with one or more cleaved iCaspase substrates. In some embodiments, the library is positively selected by incubating with one or more peptides comprising P4-P3-P2-D at the C-terminus. In some embodiments, P4 is a hydrophobic amino acid. In some embodiments, P4 is not D. In some embodiments, P3 is E, V, or Q. In some embodiments, P2 is H, S, or T.

In some embodiments, multiple rounds of positive selection are performed with different cleaved peptides in each round. In some embodiments, multiple rounds of positive selection are performed with the same peptides in each round.

In some embodiments, the antibody library is negatively selected for antibodies that bind to a peptide comprising the amino acid sequence D-X-X-D at the C-terminus. In some embodiments, the negative selection comprises incubating the antibody with peptides comprising D-X-X-D at the C-terminus that are bound to a solid substrate and retaining the supernatant and discarding the bound antibodies. In some embodiments, the negative selection comprises incubating the antibody library with free peptides comprising D-X-X-D at the C-terminus.

In some embodiments, the positive and negative selection are simultaneous. For example, in some embodiments, the antibody library is incubated with one or more peptides comprising P4-P3-P2-D at the C terminus, wherein P4 is not D, that are bound to a solid substrate and incubated with one or more unbound peptide comprising D-X-X-D at the C-terminus. In some embodiments, P4 is a hydrophobic amino acid. In some embodiments, P3 is E, V, or Q. In some embodiments, P2 is H, S, or T. In some embodiments, antibodies bound to the solid substrate are selected.

In some embodiments, the positive and negative selection are simultaneous. For example, in some embodiments, the antibody library is incubated with one or more unbound peptides comprising P4-P3-P2-D at the C terminus, wherein P4 is not D, that are bound to a solid substrate and incubated with one or more peptides comprising D-X-X-D at the C-terminus that are bound to a solid substrate. In some embodiments, P4 is a hydrophobic amino acid. In some embodiments, P4 is W, I, L, or Y. In some embodiments, P3 is E, V, or Q. In some embodiments, P2 is H, S, or T. In some embodiments, antibodies not bound to the solid substrate are selected.

In some embodiments, the positive and negative selection are sequential. For example, in some embodiments, the antibody library is first negatively selected for antibodies that bind to peptides comprising D-X-X-D at the C-terminus and then positively selected for antibodies that bind to peptides comprising P4-P3-P2-D at the C-terminus. In some embodiments, the antibody library is first positively selected for antibodies that bind to peptides comprising P4-P3-P2-D at the C-terminus and then negatively selected for peptides comprising D-X-X-D at the C-terminus. In some embodiments, P4 is a hydrophobic amino acid. In some embodiments, P4 is W, I, L, or Y. In some embodiments, P3 is E, V, or Q. In some embodiments, P2 is H, S, or T.

In some embodiments, the antibody library is negatively selected for antibodies that bind to a peptide comprising the amino acid sequence E-X-X-D at the C-terminus. In some embodiments, the negative selection comprises incubating the antibody with peptides comprising E-X-X-D at the C-terminus that are bound to a solid substrate and retaining the supernatant and discarding the bound antibodies. In some embodiments, the negative selection comprises incubating the antibody library with free peptides comprising E-X-X-D at the C-terminus.

In some embodiments, the positive and negative selection are simultaneous. For example, in some embodiments, the antibody library is incubated with one or more peptides comprising P4-P3-P2-D at the C terminus, wherein P4 is not D, that are bound to a solid substrate and incubated with one or more unbound peptide comprising E-X-X-D at the C-terminus. In some embodiments, P4 is a hydrophobic amino acid. In some embodiments, P3 is E, V, or Q. In some embodiments, P2 is H, S, or T. In some embodiments, antibodies bound to the solid substrate are selected.

In some embodiments, the positive and negative selection are simultaneous. For example, in some embodiments, the antibody library is incubated with one or more unbound peptides comprising P4-P3-P2-D at the C terminus, wherein P4 is not D, that are bound to a solid substrate and incubated with one or more peptides comprising E-X-X-D at the C-terminus that are bound to a solid substrate. In some embodiments, P4 is a hydrophobic amino acid. In some embodiments, P4 is W, I, L, or Y. In some embodiments, P3 is E, V, or Q. In some embodiments, P2 is H, S, or T. In some embodiments, antibodies not bound to the solid substrate are selected.

In some embodiments, the positive and negative selection are sequential. For example, in some embodiments, the antibody library is first negatively selected for antibodies that bind to peptides comprising E-X-X-D at the C-terminus and then positively selected for antibodies that bind to peptides comprising P4-P3-P2-D at the C-terminus. In some embodiments, the antibody library is first positively selected for antibodies that bind to peptides comprising P4-P3-P2-D at the C-terminus and then negatively selected for peptides comprising E-X-X-D at the C-terminus. In some embodiments, P4 is a hydrophobic amino acid. In some embodiments, P4 is W, I, L, or Y. In some embodiments, P3 is E, V, or Q. In some embodiments, P2 is H, S, or T.

In some embodiments, multiple rounds of positive and negative selection are performed. For example, in some embodiments, at least two rounds, at least three rounds, at least four rounds, or at least five rounds of positive and negative selection are performed.

In some embodiments, selected antibodies are assayed to confirm that they bind to peptides comprising P4-P3-P2-D at the C-terminus but not D-X-X-D at the C-terminus. In some embodiments, the antibodies are assayed using ELISA or SPR.

Also provided herein are antibodies produced by the methods of screening provided herein.

D. Methods of Detection

Also provided herein is a method of detecting cleavage of an iCaspase substrate in a sample comprising contacting the sample with an anti-cleaved iCaspase substrate antibody provided here and detecting a cleaved iCaspase substrate.

In some embodiments cleavage of an iCaspase substrate is detected in a blood, plasma, serum, urine, saliva, sputum, lung effusion, or a tissue sample. In some embodiments, the sample is a human sample.

In certain embodiments, the anti-cleaved iCaspase substrate antibody is conjugated with a first detectable label. Any suitable detectable labels may be used. In certain embodiments, the detectable label is a fluorescent label, such as for example, fluorophore AF-488, derivatives of cyanine dyes, fluorescein, rhodamine, Texas red, aminomethylcoumarin (AMCA), phycoerythrin, fluorescein isothiocyanante (FITC), among others. Examples of suitable labels are those numerous labels known for use in immunoassay, including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵, ³H, and ¹³¹I, fluorophores such as rare-earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, HRP, alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-p-galactosidase with MUG), spin labels, bacteriophage labels, stable free radicals, and the like. In some embodiments, the label is selected from the group consisting of biotin, digoxigenin, and fluorescein. Methods of conjugating an antibody with a detectable label are well known in the art, see for example, Hermanson, G. T., Bioconjugate techniques, Academic Press, 2008.

In certain embodiments, the anti-cleaved iCaspase substrate antibody is not conjugated. The un-conjugated anti-cleaved iCaspase substrate antibody can be detected with a secondary antibody conjugated with a detectable label (e.g. the first detectable label). Such secondary antibody can be any antibody raised in a different species than the anti-cleaved iCaspase substrate antibody and recognizes the constant region of the anti-cleaved iCaspase substrate antibody, as is commonly employed in the art.

The detection can be carried out by any suitable method, for example, those based on immunofluorescent microscopy, flow cytometry, fiber-optic scanning cytometry, or laser scanning cytometry. In some embodiments, the detection is an immunoassay. In some embodiments, the detection is an enzyme linked immunosorbent assay or radioimmunoassay. In some embodiments, the immunoassay comprises immunoblotting, immunodiffusion, immunoelectrophoresis, or immunoprecipitation. In some embodiments, a cleaved iCaspase substrate is detected by blotting with an anti-cleaved iCaspase substrate antibody

Also provided herein is a method of enriching cleaved iCaspase substrates in a sample. This method is especially useful for identifying previously unknown iCaspase substrates. In some embodiments, the method comprises contacting a mixture of polypeptides with an anti-cleaved iCaspase substrate antibody as provided herein, and selecting antibody-bound polypeptides from the sample. In some embodiments, the anti-cleaved iCaspase substrate antibody is bound to a solid support. In some embodiments, the selection comprises immunoprecipitation.

In some embodiments, the method further comprise identifying an iCaspase substrate from the enriched cleaved iCaspase substrates. In some embodiments, the iCaspase substrate is identified by mass spectrometry. In some embodiments, tandem mass spectrometry is performed. In some embodiments the iCaspase substrate is identified by Western Blotting. In some embodiments, the iCaspase substrate is identified by protein sequencing.

E. Identified iCaspase Substrates

Also provided herein is a method of detecting the activation of the inflammasome. In another aspect, provided herein is a method of detecting pyroptosis. In some embodiments, the method comprising measuring the abundance of an iCaspase substrate. In some embodiments, an abundance of the iCaspase substrate above a threshold indicates activation of the inflammasome. In some embodiments, an abundance of the iCaspase substrate above a threshold indicates pyroptosis. In some embodiments, the iCaspase substrate is a protein with a peptide that is immunoprecipitated by CJ11 that is greater than two-fold enriched upon LPS stimulation. In some embodiments, the iCaspase substrate is any one of the proteins listed in Table 6.

In some embodiments, the iCaspase substrate is 2AAA, 2ABA, A4, ABCA8, ABCF3, ABL2, ACPH, ACSL3, ADRM1, AFF4, AHNK, AKIB1, AKIR2, ANKAR, AP2B1, API5, APOL2, ARMC6, ASM3A, ASUN, ATLA3, ATPB, ATX10, B3GN2, B4DDM6, B7Z223, BAZ1A, BAZ2A, BCAT1, BCDO2, BRD7, BRD8, BRD9, BUB3, C2D1B, C9JFC0, CAF1B, CARM1, CASP6, CASP7, CBX2, CCD47, CCZ1B, CD109, CD9, CDC42, CDK12, CENPT, CEP68, CLC14, CLCA1, CLMP, CLOCK, CNDP2, CNOT1, CNOT2, COA5, COG8, COIL, CORO7, CREL2, CWC22, CXXC1, CYBP, DAPK3, DBX1, DCAF8, DCUP, DDX1, DDX10, DDX17, DDX18, DDX23, DDX5, DDX55, DDX60, DHE3, DHX9, DIDO1, DJC10, DLG5, DOP2, DPYD, DUS12, DVL3, DYHC1, DYST, ECE1, DEM3, EF1A1, EGFR, EIF2D, EIF3G, EMC1, ENPLL, ERBB2, ERF1, ERF3A, ERLN1, ERO1A, F220A, F8WEM9, FAAA, FBXL6, FGFR1, FKBP7, FLNA, FLNC, FNTB, FR10P, FRITZ, FRPD4, G3P, G6PD, GANAB, GBB1, GBB2, GBF1, GDF15, GELS, GEMI5, GFPT1, GNPI1, GPX1, GRP75, GSDMD, H0YDK2, H0YJR7, H3BQP1, H3BS87, HEBP1, HELZ2, HEM6, HEMH, HERC1, HGS, HIRP3, HJURP, HNRPK, HPRT, HS105, HS71A, HSP7C, HTRA2, HYCCI, HYI, IF2P, IF5, IGLL5, IL6RB, ILF2, IMDH1, IMDH2, IPO9, ITA1, ITM2B, JHD2C, K319L, KANL1, KAPCA, KBL, KPCA, KTN1, KTNB1, LIMK1, LMBD2, LMBL3, LOXL2, LPIN1, LPPRC, LRC41, LTN1, LTOR3, MAT2B, MCM3, MCU, METK1, MIC10, MINT, MKRN2, MON2, MPIP2, MRP1, MSPD2, MTD2L, MTMRA, MTX2, MVP, MYH9, MYO1C, NASP, NBN, NCOR2, NEBU, NEP1, NEUL, NEUR3, NFS1, NHSL1, NIBL1, NKTR, NP1L1, NPL4, NQO1, NSNSC, NT5D3, NTH, NU205, NUP43, ODO1, P4HA1, P5CS, A1B2, PACS1, PARP2, PCD12, PCY1B, PDLI5, DPR, PGBM, PHF19, PHLP1, PI3R4, PKHA5, PLEC, PLST, PP2BA, PRDX1, PRDX4, PRKRA, PRP31, PRP8, PRS7, PSA7, PSB3, PSD12, PTN2, PTPRF, PTPRG, PURA2, PUS10, PYR1, PYRG1, Q5HYG5, RAB14, RAB9A, RACK1, RAD17, RBP10, RBP2, RCAN1, RCC2, RECQ1, RHG01, RHG10, RHOQ, RL10, RM24, RPB3, RPC1, RSSA, RTCB, SAMH1, SAP18, SC22B, SC24B, SC24D, SCFD2, SDE2, SDHA, SEM6C, SEP11, SEPT2, SEPT9, SERPH, SF3A1, SF3B1, SHB, SIAE, SIGIR, SIN1, SLIT2, SNR40, SNX6, SNX9, SPAT5, SPOP, SPRY4, SPS1, SPTB1, SPTN1, SQSTM, SRBD1, SRP54, SRRT, STAR9, STIP1, SUFU, SUMO2, SYNE1, SYTL2, SYTM, T126A, T22D2, TAU, TBB2A, TBG1, TCPG, TCRG1, TEX2, TGFI1, THOC6, THOP1, THRB, TIF1B, TITIN, TMED8, TNS2, TPC11, PM1, TRI25, TRIP4, TRM13, TRRAP, TSC2, TTC13, TTC27, TTC9C, TXNL1, U2AF1, U5S1, UBA1, UBP15, UBP24, UBP7, UGPA, URB2, UTP4, VATB1, VATH, VINC, VPP1, WDR43, WDR7, XPO1, XRN2, YETS2, ZCCHV, or ZN185.

In some embodiments, the iCaspase substrate is Uniprot Accession No. P30153, P63151, P05067, O94911, Q9NUQ8, P42684, P13798, O95573, Q16186, Q9UHB7, Q09666, Q9P2G1, Q53H80, Q7Z5J8, P63010, Q9BZZ5, Q9BQE5, Q6NXE6, Q92484, Q9NVM9, Q6DD88, P06576, Q9UBB4, Q9NY97, B4DDM6, B7Z223, Q9NRL2, Q9UIF9, P54687, Q9BYV7-4, Q9NPI1, Q9H0E9, Q9H8M2, O43684, Q5T0F9, C9JFC0, Q13112, Q86X55, P55212, P55210, Q14781, Q96A33, P86790, Q6YHK3, P21926, P60953, Q9NYV4, Q96BT3, Q76N32, Q86T13, A8K7I4, Q9H6B4, O15516, Q96KP4, A5YKK6, Q9NZN8, Q86WW8, Q96MW5, P38432, P57737, Q6UXH1, Q9HCG8, Q9POU4, Q9HB71, O43293, A6NMT0, Q5TAQ9, P06132, Q92499, Q13206, Q92841, Q9NVP1, Q9BUQ8, P17844, Q8NHQ9, Q8IY21, P00367, Q08211, Q9BTC0, Q8IXB1, Q8TDM6, Q9Y3R5, Q12882, Q9UNI6, Q92997, Q14204, Q03001, P42892, Q9BZQ6, P68104, P00533, P41214, O75821, Q8N766, Q58FF3, P04626, P62495, P15170, O75477, Q96HE7, Q7Z4H9, F8WEM9, P16930, Q8N531, P11362, Q9Y680, P21333, Q14315, P49356, O95684, O95876-3, Q14CM0, P04406, P11413, Q14697, P62873, P62879, Q92538, Q99988, P06396, Q8TEQ6, Q06210, P46926, P07203, P38646, P57764, H0YDK2, H0YJR7, H3BQP1, H3BS87, Q9NRV9, Q9BYK8, P36551, P22830, Q15751, O14964, Q9BW71, Q8NCD3, P61978, P00492, Q92598, P0DMV8, P11142, O43464, Q9BYI3, Q5T013, O60841, P55010, B9A064, P40189, Q12905, P20839, P12268, Q96P70, P56199, Q9Y287, Q15652, Q8IZA0, Q7Z3B3, P17612, O75600, P17252, Q86UP2, Q9BVAO, P53667, Q68DH5, Q96JM7, Q9Y4KO, Q14693, P42704, Q15345, O94822, Q9UHA4, Q9NZL9, P25205, Q8NE86, Q00266, Q5TGZO, Q96T58 Q9H000, Q7Z3U7, P30305, P33527, Q8NHP6, Q9H903, Q9NXD2, O75431, Q14764, P35579, O00159, P49321, O60934, Q9Y618, P20929, Q92979, Q9BYT8, Q9UQ49, Q9Y697, Q5SYE7, Q96TA1, P30414, P55209, Q8TAT6, P15559, Q63ZY6, Q86UY8, P78549, Q92621, Q8NFH3, Q02218, P13674, P54886, P68402, Q6VY07, Q9UGN5, Q9NPG4, Q9Y5K3, Q96HC4, Q8NCN5, P98160, Q5T6S3-2, O60346, Q99570, Q9HAUO, Q15149, P13797, Q08209, Q06830, Q13162, O75569, Q8WWY3, Q6P2Q9, P35998, O14818, P49720, O00232, P17706, P10586, P23470, P30520, Q3MIT2, P27708, P17812, Q5HYG5, P61106, P51151, P63244, O75943, Q6VN20, P49792, P53805, Q9P258, P46063, Q07960, A1A4S6, P17081, P27635, Q96A35, P19387, O14802, P08865, Q9Y3I0, Q9Y3Z3, O00422, O75396, O95487, O94855, Q8WU76, Q6IQ49, P31040, Q9H3T2, Q9NVA2, Q15019, Q9UHD8, P50454, Q15459, O75533, Q15464, Q9HAT2, Q6IA17, Q9BPZ7, O94813-2, Q96DI7, Q9UNH7, Q9Y5X1, Q8NB90, O43791, Q8WW59, P49903, P11277, Q13813, Q13501, Q8N5C6, P61011, Q9BXP5, Q9P2P6, P31948, Q9UMX1, P61956, Q8NF91, Q9HCH5-7, Q9BW92, Q9H061, O75157, P10636, Q13885, P23258, P49368, O14776, Q8IWB9, O43294, Q86W42, P52888, P00734, Q13263, Q8WZ42, Q6PL24, Q63HR2, Q7Z392, P09493, Q14258, Q15650, Q9NUP7, Q9Y4A5, P49815, Q8NBP0, Q6P3X3, Q8N5M4, O43396, Q01081, Q15029, P22314, Q9Y4E8, Q9UPU5, Q93009, Q16851, Q14146, Q969X6, P15313, Q9UI12, P18206, Q93050, Q15061, Q9Y4E6, O14980, Q9HaD6, Q9ULM3, Q7Z2W4, or O15231.

F. Kits

The screening and detection methods of this invention can be provided in the form of a kit. In some embodiments, such a kit comprises an antibody that binds to a cleaved iCaspase substrate or a composition comprising an antibody that binds to a cleaved iCaspase substrate as described herein. In various embodiments, the antibody that binds to a cleaved iCaspase substrate is one or more of the antibodies described herein. In some embodiments, the antibody comprises a VH comprising a CDR-H1 comprising the amino acid sequence of SEQ ID NO:3, a CDR-H2 comprising the amino acid sequence of SEQ ID NO:3, and a CDR-H3 comprising the amino acid sequence of SEQ ID NO:5; and a VL comprising a CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8. In some embodiments, the antibody comprises a VH comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13; and a VL comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16. In some embodiments, the kit provides instructions for detecting a cleaved iCaspase substrate with the antibody. In some embodiments, the kit provides an anti-cleaved iCaspase substrate antibody and a method for detecting the antibody. For example, in some embodiments, the kit provides an antibody that is conjugated to a label. In some embodiments, the antibody is labeled with biotin, digoxigenin, or fluorescein. In some embodiments, the kit provides reagents for detecting a cleaved iCaspase substrate with the antibody. In some embodiments, the kit provides reagents for an ELISA to detect a cleaved iCaspase substrate with the antibody. In some embodiments, the kit provides reagents for detecting a cleaved iCaspase substrate in a Western blot with the antibody. In some embodiments, the kit provides reagents for an SPR assay to detect a cleaved iCaspase substrate with the antibody. In some embodiments, the kit provides reagents to detect a cleaved iCaspase substrate with the antibody in an immunoprecipitation.

In some embodiments, such a kit is a packaged combination including the basic elements of: a capture reagent comprised of an antibody that binds to a cleaved iCaspase substrate; a detectable (labeled or unlabeled) antibody that binds to a cleaved iCaspase substrate as described herein; and instructions on how to perform the assay method using these reagents. These basic elements are defined hereinabove. The kit may further comprise a solid support for the capture reagents, which may be provided as a separate element or on which the capture reagents are already immobilized. Hence, the capture antibodies in the kit may be immobilized on a solid support, or they may be immobilized on such support that is included with the kit or provided separately from the kit. In some embodiments, the capture reagents are coated on or attached to a solid material (for example, a microtiter plate, beads or a comb). The detectable antibodies may be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme; where the label is a fluorophore, a dye precursor that provides the detectable chromophore; and where the label is biotin, an avidin such as avidin, streptavidin, or streptavidin conjugated to HRP or β-galactosidase with MUG. The kit also typically contains an iCaspase substrate (e.g., GSDMD, IL1β, and/or IL18) as a standard as well as other additives such as stabilizers, washing and incubation buffers, and the like.

In some embodiments, a kit for use in a method of screening for an antibody that binds to a cleaved iCaspase substrate, as described herein, is provided. In some embodiments, the kit comprises any of the antibodies that bind to a cleaved iCaspase substrate, as described herein. In some embodiments, the kit provides instructions for performing positive selection (e.g., selection for binding a cleaved iCaspase substrate) or negative selection (e.g., selection for not binding D-X-X-D or E-X-X-D), as described herein. In some embodiments, the kit comprises a peptide library which can be used for producing and/or screening for antibodies that bind to a cleaved iCaspase substrate. In some embodiments, the peptide library comprises X-X-X-D, Y-X-X-D, I-X-X-D, L-X-X-D, or W-X-X-D at the C-terminus. In some embodiments, the kit comprises a peptide library which can be used for negative selection. In some embodiments, the peptide library for negative selection comprises a peptide comprising the amino acid sequence D-X-X-D at the C-terminus. In some embodiments, the peptide library for negative selection comprises a peptide comprising the amino acid sequence E-X-X-D at the C-terminus. In some embodiments, the kit provides a reagent for detecting binding of the antibody to a cleaved iCaspase substrate. In some embodiments, the kit provides a reagent for detecting binding of the antibody to the peptide library (e.g., X-X-X-D, Y-X-X-D, I-X-X-D, L-X-X-D, or W-X-X-D). In some embodiments, the kit provides a reagent for detecting binding of the antibody to the peptide library for negative selection. In some embodiments, binding of the antibody to the peptide library or the peptide library for negative selection is detected by ELISA. In some embodiments, the kit provides instructions or a reagent for an ELISA.

In some embodiments, a kit for use in a method of detecting cleavage of an iCaspase substrate, as described herein, is provided. In some embodiments, the kit comprises any of the antibodies that bind to a cleaved iCaspase substrate, as described herein. In some embodiments, the kit comprises instructions for detecting the cleavage of an iCaspase substrate. In some embodiments, binding of the antibody to an iCaspase substrate indicates that it is a cleaved iCaspase substrate. In some embodiments, the kit provides an antibody that is conjugated to a label. In some embodiments, the antibody is labeled with biotin, digoxigenin, or fluorescein. In some embodiments, the kit includes a cleaved iCaspase substrate (e.g., cleaved GSDMD, IL1β, and/or IL18) or a peptide (e.g., W-X-X-D or I-X-X-D) as a standard. In some embodiments, binding of the antibody to the cleaved iCaspase substrate is detected using any of the methods described herein. In some embodiments, binding of the antibody to the cleaved iCaspase substrate is detected in an ELISA. In some embodiments, the kit provides instructions or a reagent for an ELISA.

In some embodiments, a kit for use in a method of enriching cleaved iCaspase substrates in a sample comprising a mixture of polypeptides, as described herein, is provided. In some embodiments, the kit comprises any of the antibodies that bind to a cleaved iCaspase substrate, as described herein. In some embodiments, the kit comprises a reagent for contacting the sample with the antibody. In some embodiments, the reagent for contacting the sample with the antibody is a suitable buffer. In some embodiments, the kit comprises a reagent for selecting antibody-bound polypeptides from the sample. In some embodiments, the reagent for selecting antibody-bound polypeptides from the sample is a capture reagent, as described above. In some embodiments, the kit provides instructions for detecting the selected antibody-bound polypeptides. In some embodiments, the kit provides reagents for detecting the selected antibody-bound polypeptides. In some embodiments, the antibody-bound polypeptides are detected by protein sequencing. In some embodiments, the kit provides instructions for protein sequencing.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Generation of Polyclonal Antibodies with Pan-iCasp Specificity

The following example describes the generation of polyclonal antibody sera with binding specificity similar to that of the inflammatory caspases (iCasps 1/4/5/11).

### Materials and Methods

### Design of Peptide Libraries

Caspases selectively cleave substrates at primary sequence motifs (P4-P3-P2-P1) that contain an Asp at P1. While caspases tolerate significant sequence diversity at P2+P3, apoptotic caspases (3/6/7) prefer substrates with a P4 aspartic acid (e.g., DxxD), whereas, inflammatory caspases (iCasps) prefer substrates with a hydrophobic P4 residue (e.g., W/IxxD) (Thornberry N A, et al. J Biol Chem 1997 272(29):17907-17911; Kang S J, et al. J Cell Biol 2000 149(3):613-622). Accordingly, to generate antibodies with binding specificity similar to that of the inflammatory caspases (iCasps 1/4/5/11), target and decoy peptides were designed using the experimentally determined recognition motifs for the iCasps and apoptotic caspases (3/6/7) (Thornberry N A, et al. J Biol Chem 1997 272(29):17907-17911; Kang S J, et al. J Cell Biol 2000 149(3):613-622; Ramirez M L G, et al. J Biol Chem 2018 293(18):7058-7067) (see FIGS. 1A-1B).

Two peptide libraries were synthesized for use as target antigens: W/YxxD and I/LxxD, where P3 was an equimolar mixture of E, V, and Q and P2 was an equimolar mixture of H, S, and T (FIG. 1A). Two control peptide libraries were then synthesized in which P2 and P3 had the same degeneracy, but either the C-terminal carboxylate was capped with an amide (WxxD-NH2 or IxxD-NH2) or the P4 position was changed to D (DxxD) (FIG. 1C). These peptide libraries were synthesized using a split and mix method, and Edman sequencing confirmed the desired degeneracy of each library. Given the robust ability of rabbits to generate anti-peptide antibodies, rabbits were then immunized with the target peptide libraries, as described below (Weber J, Peng H, & Rader Exp Mol Med 2017 49(3):e305).

### Rabbit Immunizations and Immune Phage Selections

Eight New Zealand White Rabbits were immunized with peptide pool libraries conjugated to either Keyhole limpet haemocyanin (KLH) or ovalbumin (OVA). After the last immunization, selected rabbits were euthanized and the spleen and gut associated lymphoid tissue (GALT) were harvested. Total RNA extracted from the rabbit spleen and GALT was used to amplify the VH and VL repertoires individually. Using standard Gibson cloning methods, the VH and VL repertoires were assembled into single chain Fv (scFv) format and cloned into phagemid vector. As described above, several different antigens were used for selections including the BSA-conjugated peptide library (WxxD or IxxD) (see FIG. 1A), mixtures of peptides corresponding to known caspase 1/11 substrate products, or a single peptide corresponding to an individual caspase 1/11 substrate product.

Three rounds of selections, using both plate and solution panning, were done in which bound phage was eluted with 100 mM HCl, neutralized, and amplified in E coli XL1-blue (Stratagene) with the addition of M13KO7 helper phage (New England Biolabs). Selections were performed in the presence of excess decoy DxxD peptide libraries to drive specificity towards the canonical iCasp sequence motif. After selection, individual phage clones were picked and grown in 96-well deep well blocks with 2YT growth media in the presence of carbenicillin and M13KO7. After pelleting, the culture supernatants were used in phage ELISAs to screen for specificity.

### Polyclonal Antibody Enzyme-Linked Immunosorbent Assays (ELISAs)

BSA conjugated peptides (WxxD, IxxD, DxxD) and BSA alone were directly coated on MaxiSorp™ ELISA plates (Thermo Scientific) in triplicates at 10 μg/mL in PBS overnight at 4° C. Plates were blocked with 2% BSA at 20° C. for 2 hours. Serial dilutions of protein A-purified polyclonal sera starting at 100 μg/mL were shaken for 1-2 hours at 20° C. Plates were washed with PBS/Tween® 20 solution (PBST). After washing, an anti-rabbit Fc-specific HRP 2° antibody was shaken for 1 hour at 20° C. Plates were washed and developed with 3,3′,5,5′-Tetramethylbenzidine (TMB) substrate for 5 minutes and detected at 650 nm (see FIG. 1C).

Biotinylated peptides representing known cleaved caspase 1 and caspase 11 substrates were coated on neutravidin ELISA plates (Thermo Scientific) in triplicates at 10 μg/mL in PBS and incubated overnight at 4° C. Plates were washed and serial dilutions of peptide-purified polyclonal sera starting at 20 μg/mL were shaken for 1-2 hours at 20° C. ELISA plates were washed with PBST and developed as described above (see FIG. 1D).

### Western Blots

For standard western blotting analysis, cells were lysed in RIPA lysis buffer (50 mM Tris-HCl pH 7.4, 150 mM NaCl, 1 mM EDTA, lx Complete Protease Inhibitor (Roche), 1% Triton X-100, 0.1% SDS) with 1× Complete Protease Inhibitor (Roche Applied Science). Antibodies recognized Flag (M2 HRP, Sigma-Aldrich), Gsdmd (clone 17G2G9, Genentech), Caspase-7 (clone C7, Cell Signaling Technology) and β-actin.

### Results

Purified polyclonal sera (“pAbs”) from each immunized rabbit were characterized by ELISA against the peptide libraries and peptides from known caspase 1/11 substrates (GSDMD, IL-1β and IL-18) to identify the best rabbit(s) (FIGS. 1A-1B). Each pAb showed a preferential response to the target WxxD or IxxD peptide libraries but weak to no binding to the DxxD control (FIG. 1C). Several of the IxxD-immunized rabbits also bound the WxxD library. Several of the rabbit pAbs (37 and 39) showed some binding to DxxD, indicating that stringent selections during the subsequent mAb generation would be required to remove such antibodies. Consistent with the results against the degenerate peptide pools, all pAbs showed strong binding to all five human and mouse substrates (FIG. 1D).

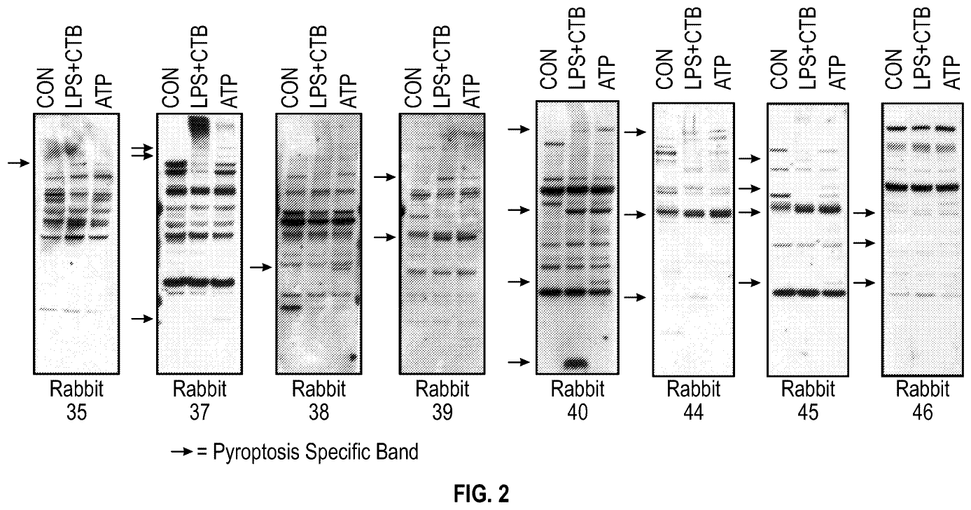

As a final and more stringent screen, western blot analyses were performed with these pAbs on lysates from mouse bone marrow derived macrophages (BMDMs) that were stimulated with two different stimuli (LPS/cholera toxin B or ATP) designed to activate the non-canonical inflammasome (Kayagaki N, et al. Nature 2011 479(7371):117-121). Encouragingly, each pAb was able to detect at least one and often multiple unique bands that was unique to the stimulated BMDM lysates (FIG. 2).

Combined, these results indicated that each immunized rabbit contained a mixture of individual monoclonal antibodies (mAbs) with a range of specificities.

### Example 2: Discovery and Characterization of Novel Pan-iCasp Monoclonal Antibody

The following example describes the generation of monoclonal antibodies with binding specificity similar to that of the inflammatory caspases.

### Materials and Methods

Monoclonal Antibody (mAb) ELISAs

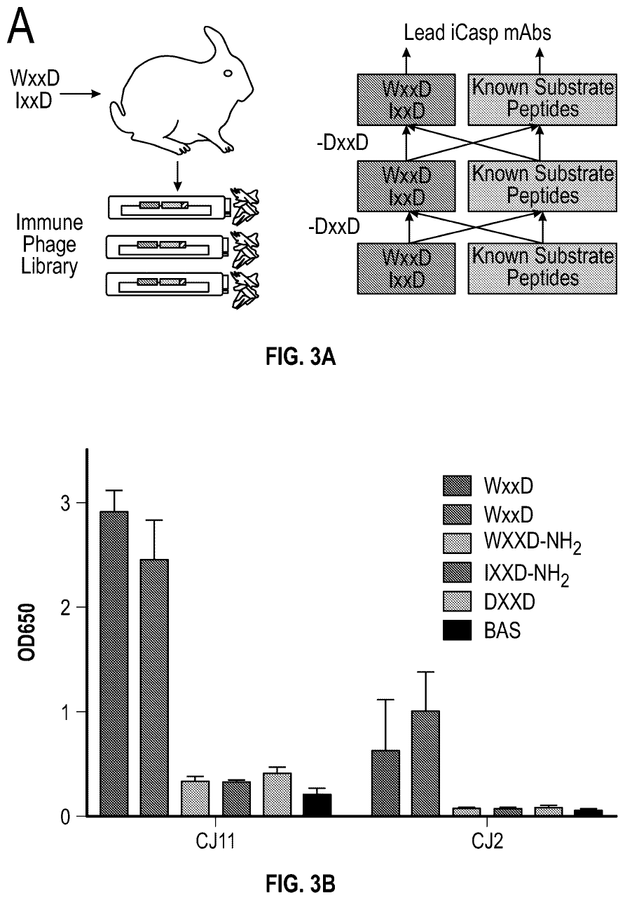

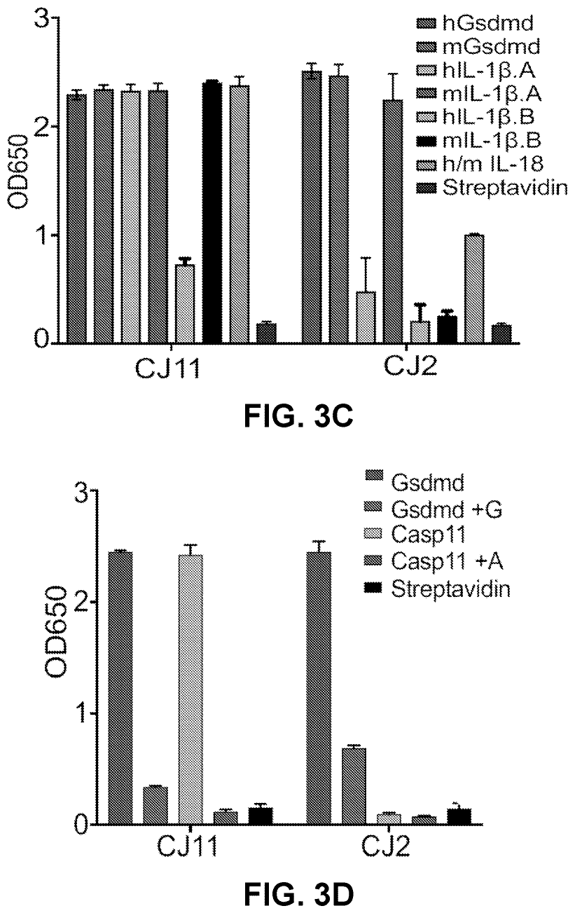

BSA-conjugated peptides (WxxD, IxxD, WxxD-NH₂, IxxD-NH₂, DxxD-W, and DxxD-I) and BSA alone were directly coated on MaxiSorp™ ELISA plates (Thermo Scientific) in triplicates at 10 μg/mL in PBS and incubated overnight at 4° C. Plates were blocked with 2% BSA for 2 hours at 20° C. Serial dilutions of CJ2 and CJ11 at 5 μg/mL were shaken for 1-2 hours at 20° C. Plates were washed and further developed as described above (see FIG. 3B).

Biotinylated peptides representing known cleaved caspase 1 and caspase 11 substrates were coated on neutravidin ELISA plates (Thermo Scientific) in triplicates at 10 μg/mL in PBS and incubated overnight at 4° C. Plates were washed and serial dilutions of CJ2 and CJ11 starting at 15 μg/mL were shaken for 1-2 hours at 20° C. ELISA plates were washed with PBST and developed as described above (see FIGS. 3C-3D).

### Specificity Profiling by Peptide Phage Display

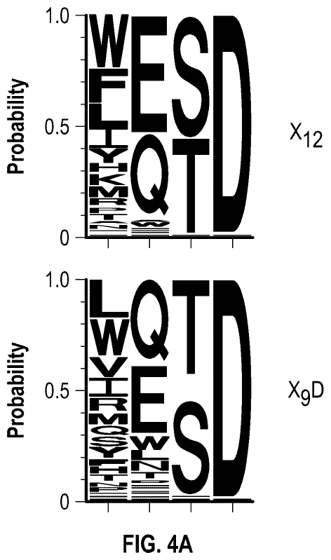

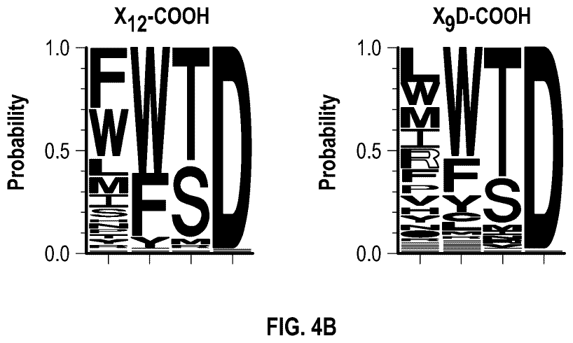

The peptide specificity of CJ2 and CJ11 was performed using p8-display libraries containing C-termini X₁₂ or X₉D peptides as previously described (Tonikian R, et al. Nat Protoc 2007 2(6):1368-1386) (see FIGS. 4A-4B).

### Monoclonal Antibody Sequencing

The amino acid sequences of antibodies CJ2 and CJ11 were determined using techniques standard in the art.

### Results

Given the diversity of antigens used for immunization, it was hypothesized that the desired monoclonal antibodies (mAbs) with iCasp specificity would be quite rare within the antibody pool. Therefore, a rabbit immune phage library strategy was designed to select for mAbs with this specificity using both W/IxxD peptide pools and individual product peptides from known iCasp substrates (FIG. 3A). Multiple phage panning tracks were performed along with stringent counter-selections against the DxxD control. Subsequent phage ELISA screens identified 423 out of 6528 that bound at least one of the target peptides. In total, fourteen unique phage clones were identified with broad recognition of both the W/IxxD peptide pools and individual substrate peptides. IgGs were generated for these clones for further characterization. Interestingly, only two out of the fourteen IgGs (CJ2 and CJ11) showed the desired P1 and P4 specificity and exhibited similar binding to both WxxD and IxxD pools by ELISA (FIG. 3B). Importantly, the mAbs showed no binding to the control peptide pools (DxxD and W/IxxD-NH₂).

Next, binding to the cleavage products from human and murine GSDMD, IL-1β, IL-18, and caspase-11 was characterized (FIGS. 3C-3D). For IL-1β, both the canonical (B) and noncanonical (A) cleavage products as defined in FIG. 1B were evaluated. Impressively, CJ11 showed strong binding to all of these peptides whereas CJ2 only exhibited binding to a subset of the targets (FIG. 3C). Addition of a single amino acid after the P1 Asp to either the GSDMD or caspase-11 peptides abolished or greatly reduced binding of both CJ2 and CJ11, indicating that direct recognition of the C-terminal carboxylate was required for high affinity binding (FIG. 3D).

Next, a peptide profiling experiment was performed to reveal the preferred recognition motifs of CJ2 and CJ11. Phage display selections against both IgGs were performed using two degenerate peptide libraries (X₁₂—COOH and X₉D-COOH, where X is any of the twenty amino acids) (Tonikian R, Zhang Y, Boone C, & Sidhu S S Nat Protoc 2007 2(6):1368-1386). In agreement with the ELISA data, both CJ11 and CJ2 only tolerated Asp at P1 and did not show recovery of any peptide with a P4 Asp (FIGS. 4A-4B). When profiled against the more diverse X₁₂ library, CJ11 enriched for peptides with predominantly hydrophobic residues at P4 with more restrictive recognition at P3 (Glu/Gln) and P2 (Ser/Thr). Interestingly, only peptides with a P1 Asp were recovered, suggesting that CJ2 and CJ11 do not recognize peptides with a highly similar P1 Glu. The X₉D library was used to more deeply profile mAb recognition within caspase-cleavage products. Here, CJ11 exhibited similar strong hydrophobic recognition at P4 with expanded diversity at P3. Therefore, two mAbs (CJ2 and CJ11) with broad recognition for known iCasp substrates that require both a free C-terminus Asp at P1 and hydrophobic residue at P4 for efficient binding were successfully isolated.

Further, the amino acid sequences of the CDRs, heavy and light chain variable regions, and full-length heavy and light chains of CJ2 and CJ11 were determined, and are provided in Tables 1-3, below.

TABLE 1
CJ2 and CJ11 heavy chain and light chain
variable region amino acid sequences

| | Vari- | | |
|---|---|---|---|
| Anti- | able | SEQ | |
| body | Re- | ID | |
| ID | gion | NO: | Amino Acid Sequence |
| CJ11 | VH | 1 | QSVEESGGGLVTPGTPLTLTCTVSGIDLSRYAMS |
| | | | WVRQAPGKGLEWIGIFGSLGGIFYASWAKGRFTI |
| | | | SKTSPTTVDLKITSPTTEDTATYFCARMPYTTDR |
| | | | DFWGPGTLVTVSS |
| CJ11 | VL | 2 | DIVMTQTPSSTSAAVGGTVTITCQASQSVANNNY |
| | | | LKWYQQKRGQPPKQLIYSVSTLASGVPSRFKGSG |
| | | | SGTQFTLTISDLECDDAATYYCSGYFNNNIGAFG |
| | | | GGTKLEIK |
| CJ2 | | 9 | QTVKESGGRLVTPGTPLTLTCTVSGIDLTTYAMT |
| | | | WVRQAPGKGLEWIGIIASSDDTNYASWAKGRFTI |
| | | | YKTSSTTVDLSITSPTTEDTATYFCARMPYTTDR |
| | | | DIWGPGTLVTVSS |
| CJ2 | VL | 10 | DVVMTQTPSSVSAAVGGTVTISCQASQSVALNSY |
| | | | LKWYQQKPGQPPKQLIYSVSTLASGVPSRFKGSG |
| | | | SGTQFTLTISDLECDDAATYYCSGYFNGNIGAFG |
| | | | AGTKLEIK |

TABLE 2
CJ2 and CJ11 complementarity-determining
region (CDR) amino acid sequences

| | | SEQ | |
|---|---|---|---|
| Antibody | | ID | Amino Acid |
| ID | CDR | NO: | Sequence |
| CJ11 | CDR H1 | 3 | RYAMS |
| CJ11 | CDR H2 | 4 | IFGSLGGIFYASWAK |
| CJ11 | CDR H3 | 5 | MPYTTDRDF |
| CJ11 | CDR L1 | 6 | QASQSVANNNYLK |
| CJ11 | CDR L2 | 7 | SVSTLAS |
| CJ11 | CDR L3 | 8 | SGYFNNNIGA |
| CJ2 | CDR H1 | 11 | TYAMT |
| CJ2 | CDR H2 | 12 | IIASSDDTNYASWAK |
| CJ2 | CDR H3 | 13 | MPYTTDRDI |
| CJ2 | CDR L1 | 14 | QASQSVALNSYLK |
| CJ2 | CDR L2 | 15 | SVSTLAS |
| CJ2 | CDR L3 | 16 | SGYFNGNIGA |

TABLE 3
CJ2 and CJ11 full-length heavy chain and light
chain amino acid sequences

| Anti- | | SEQ | |
|---|---|---|---|
| body | | ID | |
| ID | Chain | NO: | Amino Acid Sequence |
| CJ11 | Heavy | 17 | QSVEESGGGLVTPGTPLTLTCTVSGIDLSRYAMSWV |
| | chain | | RQAPGKGLEWIGIFGSLGGIFYASWAKGRFTISKTS |
| | | | PTTVDLKITSPTTEDTATYFCARMPYTTDRDFWGPG |
| | | | TLVTVSSASTKGPSVFPLAPCCGDTPSSTVTLGCLV |
| | | | KGYLPEPVTVTWNSGTLINGVRTFPSVRQSSGLYSL |
| | | | SSVVSVTSSSQPVTCNVAHPATNTKVDKTVAPSTCS |
| | | | KPTCPPPELLGGPSVFIFPPKPKDTLMISRTPEVTC |
| | | | VVVDVSQDDPEVQFTWYINNEQVRTARPPLREQQFN |
| | | | STIRVVSTLPIAHQDWLRGKEFKCKVHNKALPAPIE |
| | | | KTISKARGQPLEPKVYTMGPPREELSSRSVSLTCMI |
| | | | NGFYPSDISVEWEKNGKAEDNYKTTPAVLDSDGSYF |
| | | | LYSKLSVPTSEWQRGDVFTCSVMHEALHNHYTQKSI |
| | | | SRSPGK |
| CJ11 | Light | 18 | DIVMTQTPSSTSAAVGGTVTITCQASQSVANNNYLK |
| | chain | | WYQQKRGQPPKQLIYSVSTLASGVPSRFKGSGSGTQ |
| | | | FTLTISDLECDDAATYYCSGYENNNIGAFGGGTKLE |
| | | | IKGDPVAPTVLIFPPAADQVATGTVTIVCVANKYFP |
| | | | DVTVTWEVDGTTQTTGIENSKTPQNSADCTYNLSST |
| | | | LTLTSTQYNSHKEYTCKVTQGTTSVVQSFNRGDC |
| CJ2 | Heavy | 19 | QTVKESGGRLVTPGTPLTLTCTVSGIDLTTYAMTWV |
| | chain | | RQAPGKGLEWIGIIASSDDTNYASWAKGRFTIYKTS |
| | | | STTVDLSITSPTTEDTATYFCARMPYTTDRDIWGPG |
| | | | TLVTVSSASTKGPSVFPLAPCCGDTPSSTVTLGCLV |
| | | | KGYLPEPVTVTWNSGTLINGVRTFPSVRQSSGLYSL |
| | | | SSVVSVISSSQPVTCNVAHPATNTKVDKTVAPSTCS |
| | | | KPTCPPPELLGGPSVFIFPPKPKDTLMISRTPEVTC |
| | | | VVVDVSQDDPEVQFTWYINNEQVRTARPPLREQQFN |
| | | | STIRVVSTLPIAHQDWLRGKEFKCKVHNKALPAPIE |
| | | | KTISKARGQPLEPKVYTMGPPREELSSRSVSLTCMI |
| | | | NGFYPSDISVEWEKNGKAEDNYKTTPAVLDSDGSYF |
| | | | LYSKLSVPTSEWQRGDVFTCSVMHEALHNHYTQKSI |
| | | | SRSPGK |
| CJ2 | Light | 20 | DVVMTQTPSSVSAAVGGTVTISCQASQSVALNSYLK |
| | chain | | WYQQKPGQPPKQLIYSVSTLASGVPSRFKGSGSGTQ |
| | | | FTLTISDLECDDAATYYCSGYENGNIGAFGAGTKLE |
| | | | IKGDPVAPTVLIFPPAADQVATGTVTIVCVANKYFP |
| | | | DVTVTWEVDGTTQTTGIENSKTPQNSADCTYNLSST |
| | | | LTLTSTQYNSHKEYTCKVTQGTTSVVQSFNRGDC |

### Example 3: Structural Basis of Degenerate Recognition by QJ11

The following example describes the determination of the co-crystal structure of the CJ11 Fab and peptides representing the C-termini of IL-1β (LFFEVD) (SEQ ID NO: 32) and IL-18 (GDLESD) (SEQ ID NO: 33).

### Materials and Methods

### Fab and IgG Production

Constructs for bacterial expression of Fabs were generated by gene synthesis. Fabs were subsequently expressed and purified as previously described (Simmons L C, et al. Journal of immunological methods 2002 263(1-2):133-147; Lombana T N, et al. Scientific reports 2015 5:17488). Constructs for mammalian expression of IgGs were generated by gene synthesis. Plasmids encoding for the LC and HC were co-transfected into 293 cells and purified with affinity chromatography followed by SEC using standard methods (MabSelect SuRe™; GE Healthcare, Piscataway, NJ, USA).

### Crystallization and Data Collection

The CJ11 Fab was expressed and purified through standard protocols. The final buffer was 20 mM Tris pH 8, 100 mM NaCl, and exchanged over size exclusion chromatography. The peak fraction containing CJ11 was concentrated to 10 mg/ml, and the IL-1β peptide (₂₁LFFEVD₂₆) (SEQ ID NO: 32) was added at 1:1 molar ratio. Following incubation on ice, the protein sample was mixed in a 1:1 (v/v) ratio with mother liquor and set up in a sitting-drop vapor diffusion format over well solution containing 0.2 M calcium acetate, 0.1M sodium cacodylate pH 6.5, and 18% polyethylene glycol 8000 at 19° C. For cryoprotection, crystals were passed through reservoir solution containing 20% glycerol before flash freezing in liquid nitrogen. Diffraction data sets were collected with CMCF-081D detector at Canadian Light Source. In order to obtain crystals of CJ11 Fab complexed with IL-18 (₃₀GDLESD₃₅) (SEQ ID NO: 33), the Fab was first modified by reductive lysine methylation using standard protocols, then mixed with IL-18 peptide at 1:1 molar ratio (Walter T S, et al. Structure 2006 14(11):1617-1622). Following incubation on ice, the protein sample was mixed in a 1:1 (v/v) ratio with mother liquor containing 0.1 M sodium citrate pH 6, 20% polyethylene glycol 4000, 20% isopropanol and set up in a sitting-drop vapor diffusion format at 19° C. Crystals were flash frozen in liquid nitrogen with reservoir solution as cryoprotectant. Diffraction data sets were collected with ALS 5.0.2 detector at Advanced Light Source.

### Structure Determination and Refinement

X-ray diffraction data were integrated and scaled using HKL2000 (Otwinowski Z & Minor W Methods Enzymol 1997 276:307-326). CJ11-IL-1β crystallized in the P2₁ space group with two complexes in the asymmetric unit. The structure was determined by molecular replacement using PHENIX with a Fab search model (PDB: 5I8O) (Adams P D, et al. Acta Crystallogr D Biol Crystallogr 2010 66(Pt 2):213-221). Following molecular replacement, clear electron density was visible for the IL-1β peptide. The model was manually rebuilt through iterative refinement and omit maps using COOT and PHENIX (Adams P D, et al. Acta Crystallogr D Biol Crystallogr 2010 66(Pt 2):213-221; Emsley P, et al. Acta Crystallogr D Biol Crystallogr 2010 66(Pt 4):486-501). Secondary structure restraints were initially applied during refinement but relaxed, and TLS parameters were also tested but not employed. The IL-1β peptide was modeled only at very late stages of refinement after all protein and most solvent molecules were accounted for.

CJ11-IL-18 crystallized in the P1 space group with eight complexes in the asymmetric unit. Translational pseudo-symmetry appeared to be present and was pathological across multiple data sets collected. Ultimately, refinement statistics and map quality was consistently best when treated in P1 using the twin operator h,-k, -h-l. Diffraction data were highly anisotropic, leading to poor overall completeness in P1, and data were reduced using the anisotropy server (Strong M, et al. Proc Natl Acad Sci USA 2006 103(21):8060-8065). The structure was determined by molecular replacement using PHENIX using the previously built and refined CJ11 Fab search model after peptide and waters were removed from the model, and B-factors were flattened (Adams P D, et al. Acta Crystallogr D Biol Crystallogr 2010 66(Pt 2):213-221). Following molecular replacement, clear electron density was visible for the IL-18 peptide. NCS and secondary structure restraints were initially applied during refinement but relaxed, and TLS parameters were also tested but not employed. The IL-18 peptide was modeled only at very late stages of refinement after all protein and most solvent molecules were accounted for. Although the structure was ultimately refined to 2.45 Å, data extending to 1.70 Å was used to calculate and expect maps, and also used for realspace refinement in COOT (Emsley P, et al. Acta Crystallogr D Biol Crystallogr 2010 66(Pt 4):486-501). All structural figures were generated using PyMOL (Schrodinger LLC, The PyMOL molecular graphics system Version 2.0, Schrodinger LLC, New York, NY, 2017).

### Results

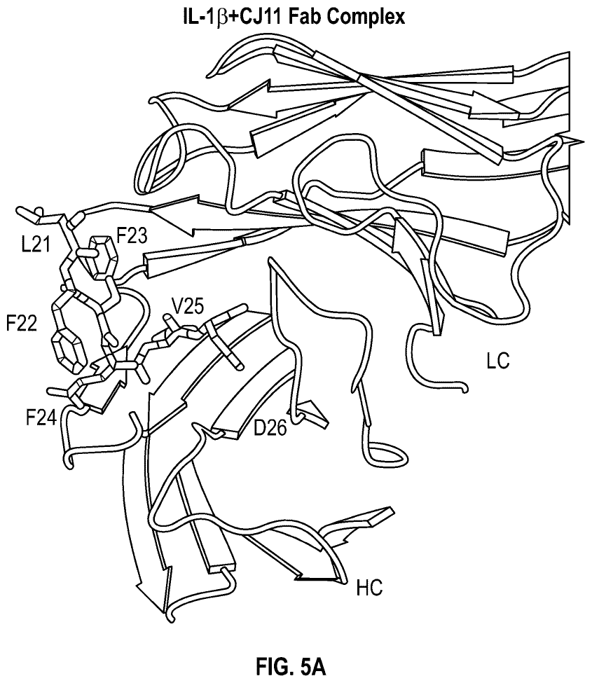

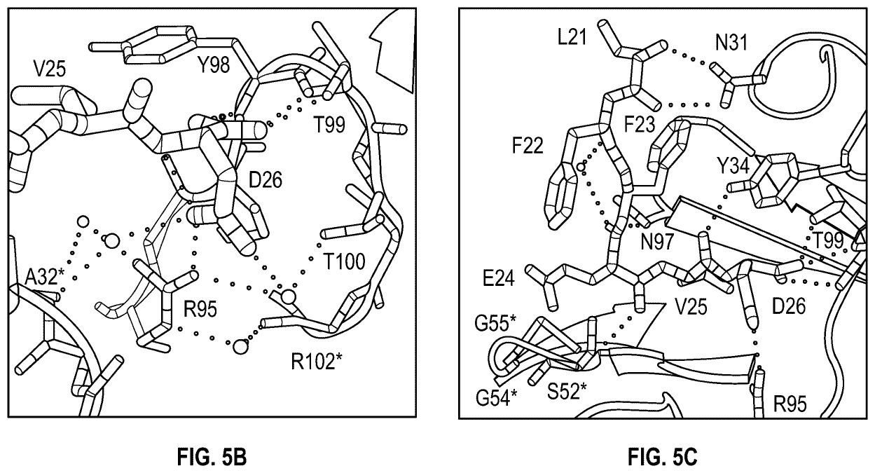

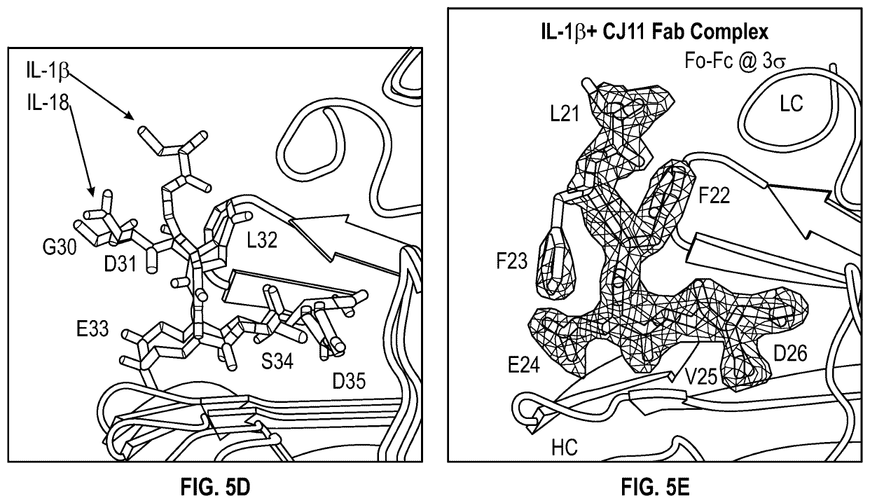

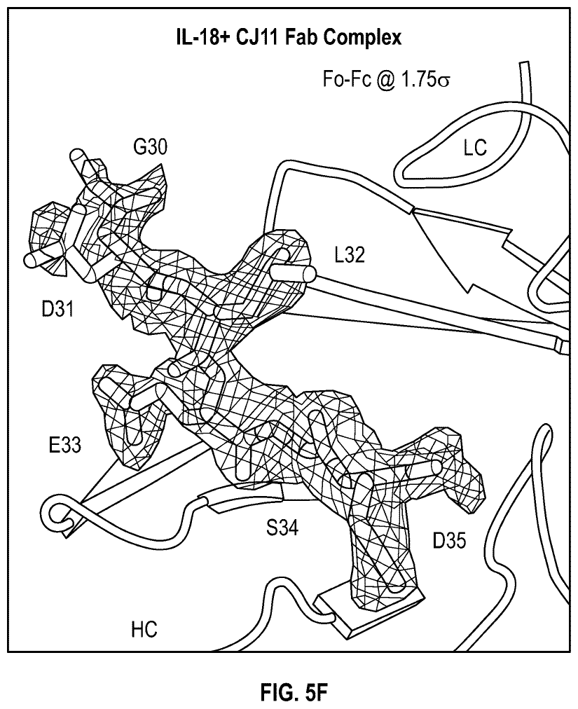

To illuminate the basis of the unusual specificity of CJ11, co-crystal structures were determined of its Fab with peptides representing the liberated C-termini of mouse IL-1β (LFFEVD) (SEQ ID NO: 32) and IL-18 (GDLESD) (SEQ ID NO: 33) to 2.0 and 3.0 Å resolution, respectively (Table 4). In both CJ11-IL-1β and IL-18 complexes, CJ11 targeted the free C-terminal carboxylic and aspartic acid groups while simultaneously forming an array of main-chain interactions along the peptides (FIGS. 5A-5F). Without wishing to be bound by theory, these observations may explain how CJ11 achieved a high selectivity in the context of a degenerate consensus motif. Below, the CJ11-IL-1β complex structure is discussed in detail, since it was of the highest resolution.

TABLE 4
Crystallographic data collection and refinement statistics.

| | |
|---|---|
| CJ11-IL-1β (₂₁LFFEVD₂₆) | CJ11-IL-18 (₃₀GDLESD₃₅) |
| (SEQ ID NO: 32) | (SEQ ID NO: 33) |

Data collection

| Space group | P21 | | P1 | |
|---|---|---|---|---|
| Cell dimensions | | | | |
| a, b, c (Å) | 66.04, 108.55, 65.9 | | 81.35, 83.52, 145.8 | |
| α, β, γ (°) | 90, 90.03, 90 | | 107.24, 106.52, 89.98 | |
| Resolution (Å) | 30-2.00 (2.44-2.37; 2.03-2.00)* | | 25-1.7 (3.09-2.91; 1.72-1.70) | |
| R_{sym} or R_{merge} | 0.084 | (0.331; 0.681) | 0.09 | (0.096; 0.505) |
| I/σI | 11.97 | (2.6; 1.1) | 6.5 | (8.1; 0.69) |
| Completeness (%) | 86 | (92.7; 45.6) | 64.6 | (79.5; 26.7) |
| Redundancy | 2.8 | (2.7; 1.8) | 1.4 | (1.4; 1.1) |

Refinement

| | | |
|---|---|---|
| Resolution (Å) | 29.5-2.00 | 23.4-3.0 |
| No. reflections | 54,077 | 60,389 |
| R_{work}/R_{free} ^{a} | 20.57/23.48 | 26.14/28.13 |
| No. atoms ^{b} | 6,967 | 25,753 |
| Fab + peptide | 6,494 | 25,753 |
| Water | 473 | n/a |
| B-factors | 41.9 | 22.5 |
| Fab | 41.8 | 22.4 |
| Peptide | 39.1 | 32.2 |
| Water | 43.3 | n/a |
| R.m.s. deviations | | |
| Bond lengths (Å) | 0.003 | 0.005 |
| Bond angles (°) | 0.64 | 1.00 |

*Values in parentheses are for a representative intermediate as well as the highest-resolution shell. Single crystals were collected for each data set.
CJ11-IL-1β Ramachandran plot: 96.94% favored, 3.06% allowed, 0% outlier.
CJ11-IL-18 Ramachandran plot: 96.8% favored, 2.94% allowed, 0.27% outlier.
^{a} 5% of reflections used to calculate R_{free}.
^{b} represents all non-hydrogen atoms modeled.

### CJ11-IL-1β Co-Crystal Structure

The IL-1β ₂₁LFFEVD₂₆ (SEQ ID NO: 32) motif adopted an “L-shape” that docked onto a strong electropositive surface patch at the intersection between the heavy chain (HC) and light chain (LC) of CJ11 (FIG. 5A, 5E). The free acidic terminus of IL-1β (Asp26) directly engaged the backbone amides of HC-Tyr98 and HC-Thr99 from complementarity-determining region 3 (CDR3), while the Thr99 hydroxyl also formed a hydrogen-bonding interaction (FIG. 51B). Without wishing to be bound by theory, these close-fitting interactions explained why an extension of, or modification to, the carboxy terminus of the peptide was not compatible with CJ11 binding. Orthogonally, the acid side-chain of Asp26 formed a tight ionic interaction with the guanidino group of HC-Arg95, and also bound to the HC-Thr100 side-chain and HC-Ala32 backbone amide through water mediated interactions (FIG. 5B). Three structural observations suggested why an aspartic acid, and not a glutamic acid, was so strongly preferred by CJ11. First, Asp26 formed a direct hydrogen bond to its own amide backbone to stabilize and orient the side-chain for productive CJ11 binding (FIG. 5B), whereas a similar coordination scheme would be geometrically disfavored by a longer glutamic acid. Second, HC-Arg95 of CJ11 was itself intimately coordinated to the Fab scaffold through a surrounding water network, suggesting that the guanidine group is optimally pre-positioned to bind an aspartic acidic (FIG. 5B). Third, the overall snug-fit of both the carboxylic acid and side-chain of Asp26 implied a precise lock-and-key recognition event that would sterically preclude the larger glutamic acid residue from binding (FIGS. 5A-5C). Thus, CJ11 organized a multipoint network to form 6 interactions with Asp26 of IL-1β through a distinctive arrangement that, without wishing to be bound by theory, explained its high selectivity to bind terminal aspartic acid residues (FIG. 5B).

To optimally position the terminal Asp26 of IL-1β for binding, CJ11 coordinated the five preceding residues primarily by exploiting the peptide backbone (FIG. 5C). The amide and carbonyl of Leu21 were both bound directly by LC-Asn31 (CDR1); the Phe22 carbonyl and Phe23 amide were coordinated to LC-Asn97 (CDR3) through water molecules; the Glu24 carbonyl interacted with the backbone amide of HC-Ser52 (CDR2); and the Val25 carbonyl hydrogen-bonds to the hydroxyl of LC-Try34 (CDR1) (FIG. 5C). This elaborate backbone coordination scheme most likely explained the degenerate nature of the CJ11 consensus binding motif. In fact, Leu21 and Phe22 side-chains are completely solvent exposed, indicating degeneracy at these positions, and rationalizing why Gly30 and Asp31 from IL-18 (₃₀GDLESD₃₅) (SEQ ID NO: 33) were permissive for CJ11 binding (FIGS. 5C-5D, 5F). The IL-1β Phe23 and Val25 side-chains bound into a small or large polar cleft on CJ11, respectively, but were not strict binding determinants because Leu32 and Ser34 within the IL-18-CJ11 complex highlighted the permissive nature of these side-chain docking clefts on CJ11 (FIG. 5D). Notably, relative to Phe23 on IL-1β, Leu32 of IL-18 packed against CJ11 in a manner that enforced a distinct local backbone geometry, which served to direct Asp31 and Gly30 towards solvent (FIG. 5D). Beyond Asp26, Glu24 was the only side-chain from IL-1β to interact specifically with CJ11, and made hydrogen bonds to the backbone amides of HC-Gly54 and HC-Gly55 (CDR2) (FIG. 5C). However, the Glu24 side-chain was also largely solvent exposed, which rationalized the lack of strict consensus for CJ11 binding, but the preference for a glutamic acid or glutamine at this position. While the noncanonical IL-1β ₂₁LFFEVD₂₆ (SEQ ID NO: 32) cleavage site was used for the structural analysis described herein, structural modeling using the canonical ₁₁₁EAYVHD₁₁₆ (SEQ ID NO: 41) cleavage site indicated that this peptide would also be capable of binding CJ11, as shown in FIGS. 3A-3D. The terminal Asp was completely conserved between both peptides. It is anticipated that the change from Val to His at P2 would maintain the backbone recognition and the side chain is readily accommodated by the surrounding polar environment. The change from Glu to Val at P3 is also be expected to be tolerated since it is partially solvent exposed and the Val would also make favorable van der Waals interactions. The other changes at P4-P6 occur at highly exposed positions, which enable degenerate recognition.

Overall, the structural analysis presented above permitted a molecular level dissection of the unique coordination strategy that CJ11 Fab employed to selectively recognize degenerate peptide motifs that terminate with a free aspartic acid.

### Comparison of Substrate Binding by Inflammatory Caspases and CJ11

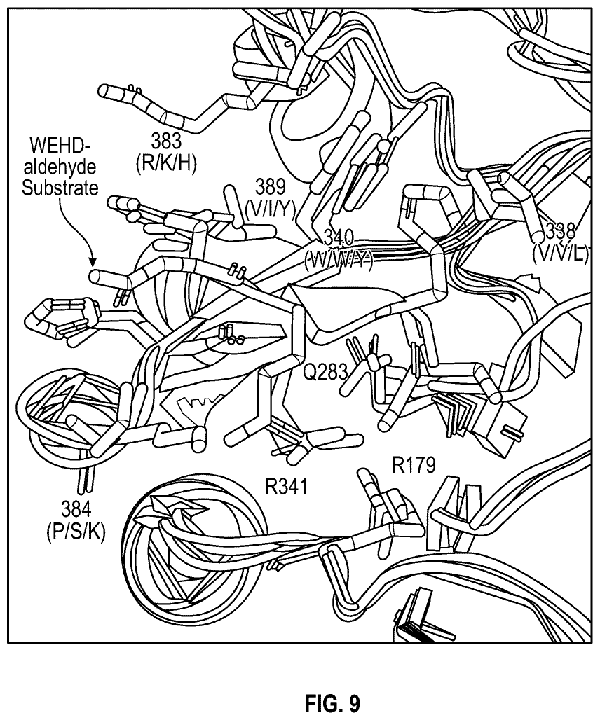

The structural basis of CJ11's recognition of inflammatory caspase substrates was compared to that of the inflammatory caspases themselves. CJ11 and the inflammatory caspases recognize similar substrates via distinct modes of molecule recognition. For caspase-1/4/11, the P1 Asp lied buried in a pocket comprised of electrostatic contacts with Arg179 and Arg341 and a hydrogen bond with Gln283 (FIG. 9). For CJ11, the P1 Asp lied in a pocket composed entirely of residues from CDRH3 that recognize both the C-terminal carboxylate through main chain amines in Tyr97 and Thr98 and the Asp side chain via ionic interaction with Arg95.

By analogy to the caspase mode of recognition, it is anticipated that mutation of HC.Thr99 or HC.Tyr32 to Arg could provide enhanced P1 Asp recognition (see Table 5). Although the P2 side chain was surface exposed upon binding to the caspase, CJ11 partially buries the P2 side chain in a pocket containing HC.Ile50 and LC.Tyr93, where these two bulky residues sterically block recognition of peptides that contain larger side chains at the P2 position. Finally, while the exact molecular basis for lack of P4 Asp/Glu recognition by CJ11 remains unclear, it is expected that a P4 Asp would be partially solvent exposed and not have steric clash or charge repulsion with the mAb. However, a side chain carboxylate at P4 might offend the adjacent carbonyl of P5, which is tightly engaged by LC.Asn31 (FIG. 5C).

TABLE 5
CJ2 and CJ11 full-length heavy chain sequences
with amino acid substitutions

| | Amino | | |
|---|---|---|---|
| Anti- | acid | SEQ | |
| body | substi- | ID | |
| ID | tution | NO: | Amino Acid Sequence |
| CJ11 | T99R | 21 | QSVEESGGGLVTPGTPLTLTCTVSGIDLSRYAMSW |
| | | | VRQAPGKGLEWIGIFGSLGGIFYASWAKGRFTISK |
| | | | TSPTTVDLKITSPTTEDTATYFCARMPYRTDRDFW |
| | | | GPGTLVTVSSASTKGPSVFPLAPCCGDTPSSTVTL |
| | | | GCLVKGYLPEPVTVTWNSGTLINGVRTFPSVRQSS |
| | | | GLYSLSSVVSVTSSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |
| CJ11 | Y32R | 22 | QSVEESGGGLVTPGTPLTLTCTVSGIDLSRRAMSW |
| | | | VRQAPGKGLEWIGIFGSLGGIFYASWAKGRFTISK |
| | | | TSPTTVDLKITSPTTEDTATYFCARMPYTTDRDEW |
| | | | GPGTLVTVSSASTKGPSVFPLAPCCGDTPSSTVTL |
| | | | GCLVKGYLPEPVTVTWNSGTLINGVRTFPSVRQSS |
| | | | GLYSLSSVVSVISSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |
| CJ11 | T99R | 23 | QSVEESGGGLVTPGTPLTLTCTVSGIDLSRRAMSW |
| | and | | VRQAPGKGLEWIGIFGSLGGIFYASWAKGRFTISK |
| | Y32R | | TSPTTVDLKITSPTTEDTATYFCARMPYRTDRDFW |
| | | | GPGTLVTVSSASTKGPSVFPLAPCCGDTPSSTVTL |
| | | | GCLVKGYLPEPVTVTWNSGTLTNGVRTFPSVRQSS |
| | | | GLYSLSSVVSVTSSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |
| CJ2 | T99R | 24 | QTVKESGGRLVTPGTPLTLTCTVSGIDLTTYAMTW |
| | | | VRQAPGKGLEWIGIIASSDDTNYASWAKGRFTIYK |
| | | | TSSTTVDLSITSPTTEDTATYFCARMPYRTDRDIW |
| | | | GPGTLVTVSSASTKGPSVFPLAPCCGDTPSSTVTL |
| | | | GCLVKGYLPEPVTVTWNSGTLTNGVRTFPSVRQSS |
| | | | GLYSLSSVVSVTSSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |
| CJ2 | Y32R | 25 | QTVKESGGRLVTPGTPLTLTCTVSGIDLTTRAMTW |
| | | | VRQAPGKGLEWIGIIASSDDTNYASWAKGRFTIYK |
| | | | TSSTTVDLSITSPTTEDTATYFCARMPYTTDRDIW |
| | | | GPGTLVTVSSASTKGPSVFPLAPCCGDTPSSTVTL |
| | | | GCLVKGYLPEPVTVTWNSGTLTNGVRTFPSVRQSS |
| | | | GLYSLSSVVSVTSSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |
| CJ2 | T99R | 26 | QTVKESGGRLVTPGTPLTLTCTVSGIDLTTRAMTW |
| | and | | VRQAPGKGLEWIGIIASSDDTNYASWAKGRFTIYK |
| | Y32R | | TSSTTVDLSITSPTTEDTATYFCARMPYRTDRDIW |
| | | | GPGTLVTVSSASTKGPSVFPLAPCCGDTPSSTVTL |
| | | | GCLVKGYLPEPVTVTWNSGTLINGVRTFPSVRQSS |
| | | | GLYSLSSVVSVTSSSQPVTCNVAHPATNTKVDKTV |
| | | | APSTCSKPTCPPPELLGGPSVFIFPPKPKDTLMIS |
| | | | RTPEVTCVVVDVSQDDPEVQFTWYINNEQVRTARP |
| | | | PLREQQFNSTIRVVSTLPIAHQDWLRGKEFKCKVH |
| | | | NKALPAPIEKTISKARGQPLEPKVYTMGPPREELS |
| | | | SRSVSLTCMINGFYPSDISVEWEKNGKAEDNYKTT |
| | | | PAVLDSDGSYFLYSKLSVPTSEWQRGDVFTCSVMH |
| | | | EALHNHYTQKSISRSPGK |

### Example 4: CJ11 Enables Selective Detection of iCasp Substrates in Cells

The following example describes experiments validating of the ability of CJ11 to detect iCasp substrates in cell lysates.

### Materials and Methods

### Immunoprecipitation Experiments

cDNAs encoding uncleaved, full-length forms and cleaved forms of IL-1β, IL-18, caspase-11, and Gsdmd were synthesized with a FLAG® Tag epitope and subcloned into pcDNA3.1/Zeo(+) (Life Technologies) for transient expression in human embryonic kidney (HEK) 293T cells. HEK293T cells were cultured overnight in 10-cm dishes at 1.2×10⁵ cells per mL, then transfected with 3 μg of plasmid using 10 μL Lipofectamine® 2000 according to manufacturer instructions (Thermo Fisher Scientific). Transfected cells were lysed 48 hours post transfection.

ER-Hoxb8-immortalized WT and Casp1 C57BL/6N mouse-derived immortalized macrophages have been previously described (Kayagaki N, et al. Science 2013 341(6151):1246-1249). Hoxb8 were maintained in RPMI 1640 medium supplemented with 10% (v/v) low-endotoxin fetal bovine serum (FBS; Omega Scientific), murine granulocyte-macrophage colony-stimulating factor (GM-CSF) (20 ng/mL, eBioscience), and 1 μM β-estradiol (Sigma-Aldrich) and were differentiated with 20% (v/v) L929-conditioned medium for 5 days at 37° C. with 5% CO₂. For stimulation, cells were primed with Pam3CSK4 (1 μg/mL, InvivoGen) for 5 hours on plates, followed by electroporation with ultra-pure LPS (E. coli O111:B4, InvivoGen). The Neon Transfection System was used for electroporation according to manufacturer's instructions (Thermo Fisher Scientific). Briefly, cells were collected and resuspended at 0.5×10⁶ cells/10 μL of RIPA buffer, and 0.5 μg LPS/1×10⁶ cells. Cells were electroporated using the 10-μL Neon tip with 1720 voltage, 10 width, 2 pulse settings and added to 5 mL media for four hours prior to cell lysis.

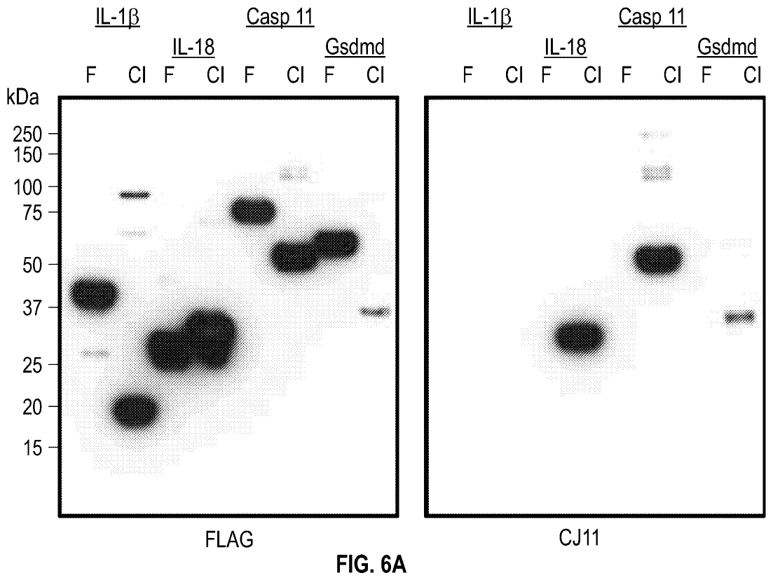

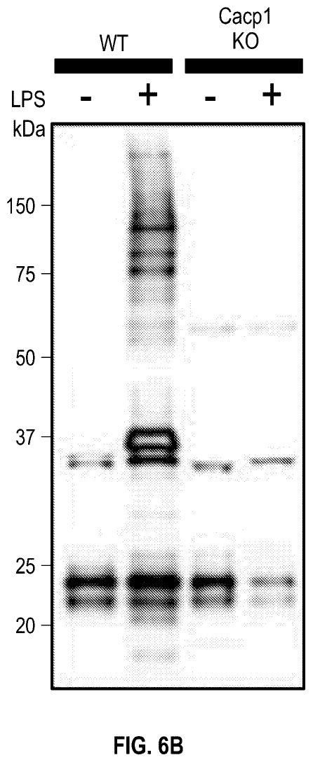

For immunoprecipitations, cells were lysed on ice for 30 minutes in IP buffer (50 mM Tris HCl pH7.4, 150 mM NaCl, 1 mM EDTA, 1% Triton™ X-100) with protease inhibitor cocktail, and lysates were cleared by centrifugation. Supernatants were incubated with 2 μg antibody (anti-Flag M2 or CJ11) and 30 μL slurry (Pierce Protein A/G Magnetic Beads) and rotated at 4° C. for 2 hours. Magnetic beads were washed four times with IP buffer and precipitates were eluted with 1×SDS sample buffer followed by SDS-PAGE and western blot (see FIGS. 6A-6B). All cell lines used were authenticated by short tandem repeat (STR) profiling, single nucleotide polymorphism (SNP) fingerprinting, and mycoplasma testing.

### Western Blots

Western blots were performed as described in Example 1, above.

### Results

Several experiments were performed to validate the ability of CJ11 to detect iCasp substrates within cell lysates. CJ11 was chosen due to its broader specificity profile. First, the ability of CJ11 to selectively immunoprecipitate the cleaved form compared to the full-length form of four known substrates (IL-1β, IL-18, caspase-11, and GSDMD) was evaluated. FLAG-tagged constructs encoding either the full-length or N-terminal fragment were expressed in HEK293 cells. Cells were then lysed and immunoprecipitations were performed with either CJ11 or anti-FLAG mAbs followed by anti-FLAG westerns for detection. Strikingly, CJ11 selectively immunoprecipitated only the cleaved form of IL-18, caspase-11, and GSDMD (FIG. 6A). The assay failed to detect any cleaved IL-1β, potentially due to its low levels of expression.

Next, an experiment was performed with immortalized mouse macrophages to gain a more complete, unbiased view of the antibody's performance (Kayagaki N, et al. Nature 2011 479(7371):117-121). Briefly, wild-type or CASP1 knockout (KO) macrophages were stimulated with cytosolic LPS to induce the presence of caspase-11 substrates. Following cell lysis, CJ11 was used to immunoprecipitate proteins and detect the proteins via Western blot. Very few bands were detected in the absence of LPS or CASP1, establishing that CJ11 exhibited very low background enrichment. Quite strikingly, CJ11 enriched for many unique bands in wild-type macrophages treated with LPS (FIG. 6B).

These results highlighted several features of CJ11. First, even in a complex cell lysate with endogenous C-termini and presumably some cleavage products from apoptotic caspases due to low levels of apoptosis, CJ11 exhibited a very high selectively for iCasp substrates. Second, activation of the inflammasome resulted in the cleavage of a multitude of substrates, which can be efficiently enriched by CJ11 immunoprecipitations.

### Example 5: Discovery of Non-Canonical Inflammasome Substrates

The following example describes the use of CJ11 to identify the non-canonical inflammasome substrates.

### Materials and Methods

Mass Spectrometry with CJ11 Immunoprecipitations (IPs)

Human EA.hy926 cells were maintained in DMEM supplemented with 10% (v/v) low-endotoxin FBS (Kayagaki N, et al. Nature 2015 526(7575):666-671). Cells were stimulated with LPS Neon™ electroporation at an LPS concentration of 0.5 μg LPS/1×10⁶ cells and electroporated using the 100-μL Neon tip with 1400 voltage, 20 width, 2 pulse settings according to manufacturer's instructions. Samples were collected one hour post Neon™ electroporation. Cells were washed once with PBS and lysed in 1 mL Urea denaturing lysis buffer (9M urea, 20 mM HEPES pH 8.0, 1 mM sodium orthovanadate. 2.5 mM sodium pyrophosphate, 1 mM β-glycerophosphate.).

iCasp substrates from human endothelial cells were enriched by immuno-affinity isolation for peptides containing motifs comprising of a C-terminal Asp and hydrophobic residues in the P4 position followed by mass spectrometry as previously described (Kim W, et al. Mol Cell 2011 44(2):325-340). Briefly, EA.hy926 lysates were normalized to 10 mg of protein (concentration based on Bradford assay), reduced at 37° C. for 1 hour in 4.5 mM DTT and alkylated with 10 mM iodoacetamide for 15 minutes at 20° C. in the dark. Samples were then diluted 4× with 20 mM HEPES pH 8.0 and sequentially digested at enzyme to protein ratios of 1:100 with Lysyl-endopeptidase (Wako Chemicals) at 37° C. for 2 hours followed by trypsin (Promega) overnight at 37° C. Following digestion, peptides were acidified and desalted using a Sep-Pak® C18 cartridge (Waters). Desalted peptides were resuspended in 1×IAP buffer (Cell Signaling Technology) and incubated with pre-coupled CJ11 antibody beads for 2 hours at 4° C. Beads were washed 2× with IAP buffer and 4× with water. Peptides were eluted off antibody resin 2× in 0.15% TFA for 10 minutes at 20° C. Immuno-affinity enriched peptides were lyophilized for 48 hrs following desalting using C₁ₛ STAGE-Tip (Rappsilber J, Ishihama Y, & Mann M Anal Chem 2003 75(3):663-670).

The desalted samples were reconstituted in 2% acetonitrile (ACN)/0.1% formic acid (FA) and subjected to LC-MS/MS analysis on an Orbitrap Fusion™ Lumos™ (Thermo) mass spectrometer coupled to a nanoAcquity® UPLC (Waters). The digested samples were loaded onto a 100 μm×250 mm PicoFrit® column (New Objective), packed with 1.7 μm BEH130 C18 (Waters) and chromatographically separated over a gradient comprising of 2-35% Buffer B (where Buffer A is 0.1% formic acid/2% acetonitrile/98% water and Buffer B is 0.1% formic acid/2% water/98% acetonitrile) at 0.5 μL/min with a total analysis time of 120 minutes. Peptides were eluted directly into the mass spectrometer and ionized at a spray voltage of 1.9 kV. Mass spectral data were acquired using a method comprising of a precursor MS1 scan (350-1350 m/z) in the Orbitrap at resolution of 120,000 followed by MS/MS spectra acquired in the LTQ of the most abundant ions subjected to HCD fragmentation (CE 30%) in a 1 second cycle time.

Tandem mass spectral results were searched using the Mascot algorithm (Matrix Science) against a concatenated target-decoy database comprised of the UniProt human proteome (version 2016_06), known laboratory contaminants and the reversed version of each sequence. A 30 ppm precursor ion mass tolerance and 0.5 Da fragment ion tolerance were selected with tryptic and aspartic acid (C-terminal) enzyme specificity and up to 2 missed cleavages. Variable modifications were allowed for carbamidomethylated cysteine residues (+57.0215 Da) and methionine oxidation (+15.9949 Da). Peptide spectral matches were filtered using LDA to an FDR of 5% at the peptide level then to an FDR of 2% at the protein level. Quantification of the peptides identified with C-terminal Asp residue at P1 position were performed using XQuant, a version of VistaGrande, for processing of unlabeled samples (Bakalarski C E, et al. J Proteome Res 2008 7(11):4756-4765; Kirkpatrick D S, et al. Proc Natl Acad Sci USA 2013 110(48):19426-19431).

Label-free quantitative data were analyzed for statistical significance using the MSStats algorithm in R (version 3.14.1; R version 3.5.3) (Choi M, et al. Bioinformatics 2014 30(17):2524-2526). Prior to statistical analysis, peptide-spectral matches (PSMs) were filtered to allow only those quantified from non-decoy proteins with a VistaGrande confidence score of 83 or greater. When multiple PSMs were identified within a MS analysis which mapped to the same peptide, the PSM with the largest area under curve (AUC) was selected. MSStats performed protein-level summarization using Tukey median polish and overall intensities were normalized using the median intensity of all AUCs quantified within an MS analysis. Differential abundance analysis between sample conditions was performed using a linear mixed-effects model per protein using MSStats as previously described and used an empirical Bayes shrinkage to adjust the inference procedure. P-values were adjusted for multiple testing using the Benjamini-Hochberg method.

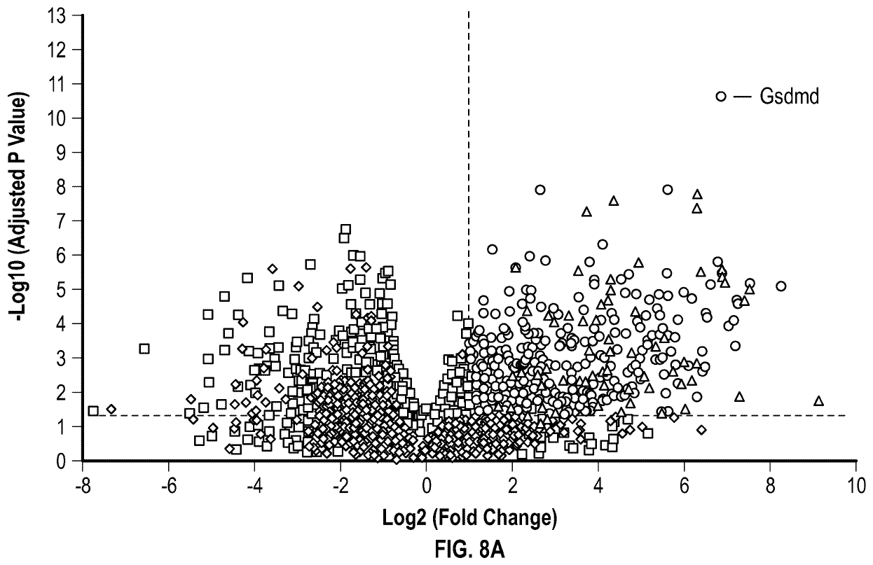

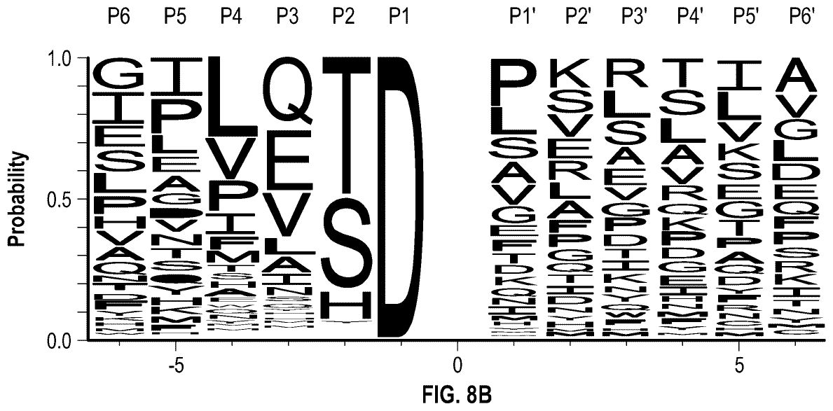

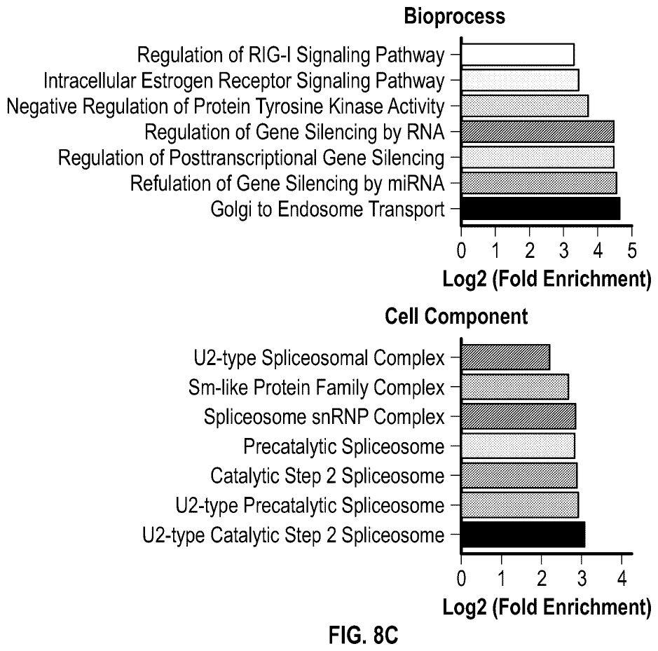

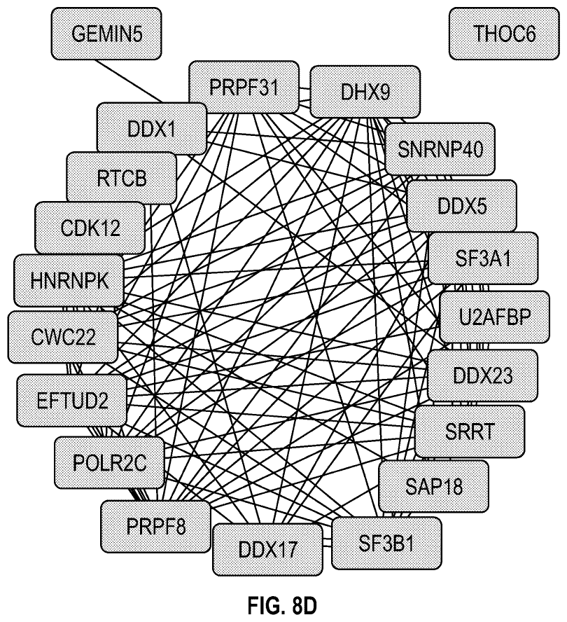

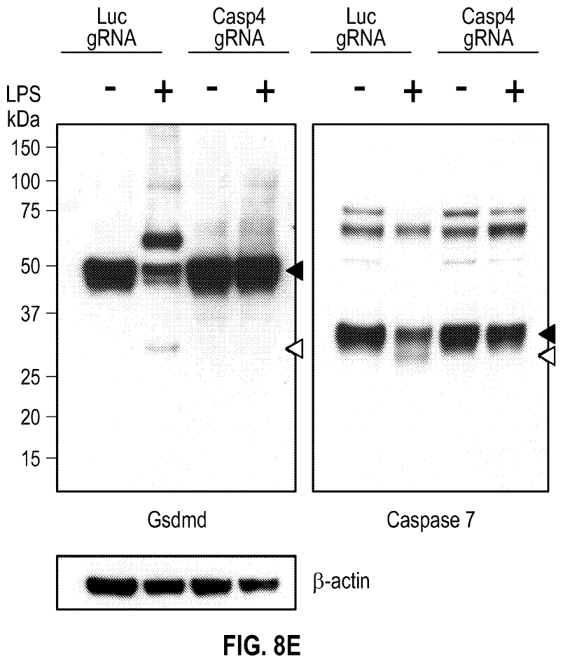

PANTHER overexpression test was used to perform GO analysis on Biological Process, Cellular Component, and Molecular Function (Thomas P D, et al. Genome Res 2003 13(9):2129-2141) (FIG. 8C). Fisher's exact test for FDR was performed and the results were filtered to include only GO terms with greater than two-fold enrichment, at least four proteins, and p<0.05. Cytoscape was used to perform protein interaction network analysis with the built-in String (Shannon P, et al. Genome Res 2003 13(11):2498-2504; Szklarczyk D, et al. Nucleic Acids Res 2019 47(D1):D607-D613) (FIG. 8D).

### Western Blots

Western blots were performed as described in Example 1, above.

### Results

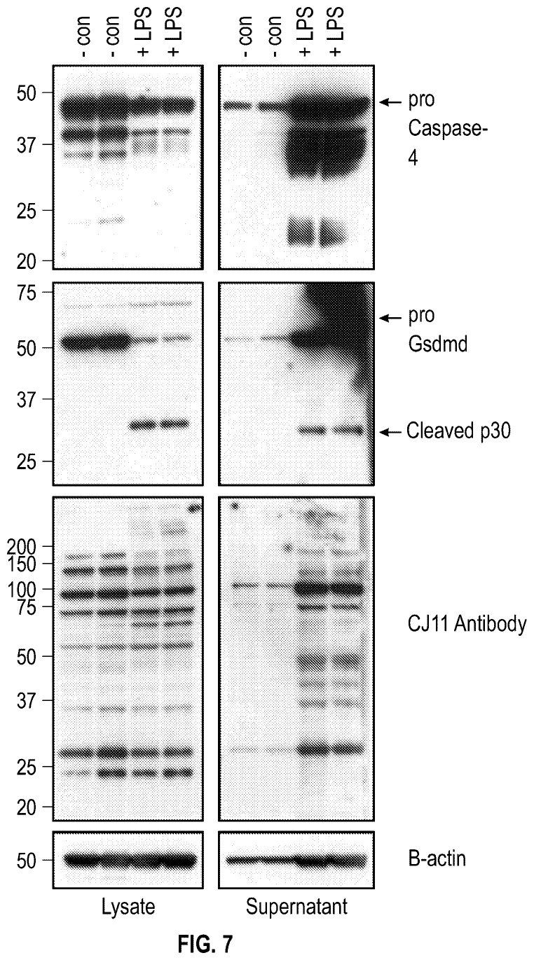

Based on the validation of CJ11 described in Example 4, whether CJ11 could be used to identify substrates of the non-canonical inflammasome and reveal potential functions beyond GSDMD-cleavage induced pyroptosis was tested. For these experiments, human EA.hy926 cells were stimulated with intracellular LPS to activate the caspase-4 non-canonical inflammasome (FIG. 7). This approach enabled a focus on substrates of the non-canonical inflammasome, since EA.hy926 cells are unable to trigger activation of the canonical inflammasome downstream of caspase-4 activation due to absence of key sensor proteins. One-hour post-stimulation, cell pellets and matched supernatants were collected. Immunoprecipitations with CJ11 followed by mass spectrometry analysis were then performed.

From both the cell lysates and matched supernatants, 4,220 unique peptides were identified. The resulting list of peptides was filtered to identify those peptides and corresponding proteins that were more than two-fold enriched upon LPS simulation (FIG. 8A) identifying 406 peptides derived from 328 proteins (Table 6). It is anticipated that these peptides could serve as novel blood-based biomarkers of inflammasome activation and, by extension, pyroptosis.

TABLE 6
List of proteins with peptides immunoprecipitated by CJ11 that
are greater than two-fold enriched upon LPS stimulation

| Protein Uniprot | Protein Uniprot |
|---|---|
| Name | Accession No. |
| 2AAA_HUMAN | P30153 |
| 2ABA_HUMAN | P63151 |
| A4_HUMAN | P05067 |
| ABCA8_HUMAN | O94911 |
| ABCF3_HUMAN | Q9NUQ8 |
| ABL2_HUMAN | P42684 |
| ACPH_HUMAN | P13798 |
| ACSL3_HUMAN | O95573 |
| ADRM1_HUMAN | Q16186 |
| AFF4_HUMAN | Q9UHB7 |
| AHNK_HUMAN | Q09666 |
| AKIB1_HUMAN | Q9P2G1 |
| AKIR2_HUMAN | Q53H80 |
| ANKAR_HUMAN | Q7Z5J8 |
| AP2B1_HUMAN | P63010 |
| API5_HUMAN | Q9BZZ5 |
| APOL2_HUMAN | Q9BQE5 |
| ARMC6_HUMAN | Q6NXE6 |
| ASM3A_HUMAN | Q92484 |
| ASUN_HUMAN | Q9NVM9 |
| ATLA3_HUMAN | Q6DD88 |
| ATPB_HUMAN | P06576 |
| ATX10_HUMAN | Q9UBB4 |
| B3GN2_HUMAN | Q9NY97 |
| B4DDM6_HUMAN | B4DDM6 |
| B7Z223_HUMAN | B7Z223 |
| BAZ1A_HUMAN | Q9NRL2 |
| BAZ2A_HUMAN | Q9UIF9 |
| BCAT1_HUMAN | P54687 |
| BCDO2_HUMAN | Q9BYV7-4 |
| BRD7_HUMAN | Q9NPI1 |
| BRD8_HUMAN | Q9H0E9 |
| BRD9_HUMAN | Q9H8M2 |
| BUB3_HUMAN | O43684 |
| C2D1B_HUMAN | Q5T0F9 |
| C9JFC0_HUMAN | C9JFC0 |
| CAF1B_HUMAN | Q13112 |
| CARM1_HUMAN | Q86X55 |
| CASP6_HUMAN | P55212 |
| CASP7_HUMAN | P55210 |
| CBX2_HUMAN | Q14781 |
| CCD47_HUMAN | Q96A33 |
| CCZ1B_HUMAN | P86790 |
| CD109_HUMAN | Q6YHK3 |
| CD9_HUMAN | P21926 |
| CDC42_HUMAN | P60953 |
| CDK12_HUMAN | Q9NYV4 |
| CENPT_HUMAN | Q96BT3 |
| CEP68_HUMAN | Q76N32 |
| CLC14_HUMAN | Q86T13 |
| CLCA1_HUMAN | A8K7I4 |
| CLMP_HUMAN | Q9H6B4 |
| CLOCK_HUMAN | O15516 |
| CNDP2_HUMAN | Q96KP4 |
| CNOT1_HUMAN | A5YKK6 |
| CNOT2_HUMAN | Q9NZN8 |
| COA5_HUMAN | Q86WW8 |
| COG8_HUMAN | Q96MW5 |
| COIL_HUMAN | P38432 |
| CORO7_HUMAN | P57737 |
| CREL2_HUMAN | Q6UXH1 |
| CWC22_HUMAN | Q9HCG8 |
| CXXC1_HUMAN | Q9P0U4 |
| CYBP_HUMAN | Q9HB71 |
| DAPK3_HUMAN | O43293 |
| DBX1_HUMAN | A6NMT0 |
| DCAF8_HUMAN | Q5TAQ9 |
| DCUP_HUMAN | P06132 |
| DDX1_HUMAN | Q92499 |
| DDX10_HUMAN | Q13206 |
| DDX17_HUMAN | Q92841 |
| DDX18_HUMAN | Q9NVP1 |
| DDX23_HUMAN | Q9BUQ8 |
| DDX5_HUMAN | P17844 |
| DDX55_HUMAN | Q8NHQ9 |
| DDX60_HUMAN | Q8IY21 |
| DHE3_HUMAN | P00367 |
| DHX9_HUMAN | Q08211 |
| DIDO1_HUMAN | Q9BTC0 |
| DJC10_HUMAN | Q8IXB1 |
| DLG5_HUMAN | Q8TDM6 |
| DOP2_HUMAN | Q9Y3R5 |
| DPYD_HUMAN | Q12882 |
| DUS12_HUMAN | Q9UNI6 |
| DVL3_HUMAN | Q92997 |
| DYHC1_HUMAN | Q14204 |
| DYST_HUMAN | Q03001 |
| ECE1_HUMAN | P42892 |
| EDEM3_HUMAN | Q9BZQ6 |
| EF1A1_HUMAN | P68104 |
| EGFR_HUMAN | P00533 |
| EIF2D_HUMAN | P41214 |
| EIF3G_HUMAN | O75821 |
| EMC1_HUMAN | Q8N766 |
| ENPLL_HUMAN | Q58FF3 |
| ERBB2_HUMAN | P04626 |
| ERF1_HUMAN | P62495 |
| ERF3A_HUMAN | P15170 |
| ERLN1_HUMAN | O75477 |
| ERO1A_HUMAN | Q96HE7 |
| F220A_HUMAN | Q7Z4H9 |
| F8WEM9_HUMAN | F8WEM9 |
| FAAA_HUMAN | P16930 |
| FBXL6_HUMAN | Q8N531 |
| FGFR1_HUMAN | P11362 |
| FKBP7_HUMAN | Q9Y680 |
| FLNA_HUMAN | P21333 |
| FLNC_HUMAN | Q14315 |
| FNTB_HUMAN | P49356 |
| FR1OP_HUMAN | O95684 |
| FRITZ_HUMAN | O95876-3 |
| FRPD4_HUMAN | Q14CM0 |
| G3P_HUMAN | P04406 |
| G6PD_HUMAN | P11413 |
| GANAB_HUMAN | Q14697 |
| GBB1_HUMAN | P62873 |
| GBB2_HUMAN | P62879 |
| GBF1_HUMAN | Q92538 |
| GDF15_HUMAN | Q99988 |
| GELS_HUMAN | P06396 |
| GEMI5_HUMAN | Q8TEQ6 |
| GFPT1_HUMAN | Q06210 |
| GNPI1_HUMAN | P46926 |
| GPX1_HUMAN | P07203 |
| GRP75_HUMAN | P38646 |
| GSDMD_HUMAN | P57764 |
| H0YDK2_HUMAN | HOYDK2 |
| H0YJR7_HUMAN | HOYJR7 |
| H3BQP1_HUMAN | H3BQP1 |
| H3BS87_HUMAN | H3BS87 |
| HEBP1_HUMAN | Q9NRV9 |
| HELZ2_HUMAN | Q9BYK8 |
| HEM6_HUMAN | P36551 |
| HEMH_HUMAN | P22830 |
| HERC1_HUMAN | Q15751 |
| HGS_HUMAN | O14964 |
| HIRP3_HUMAN | Q9BW71 |
| HJURP_HUMAN | Q8NCD3 |
| HNRPK_HUMAN | P61978 |
| HPRT_HUMAN | P00492 |
| HS105_HUMAN | Q92598 |
| HS71A_HUMAN | P0DMV8 |
| HSP7C_HUMAN | P11142 |
| HTRA2_HUMAN | O43464 |
| HYCCI_HUMAN | Q9BYI3 |
| HYI_HUMAN | Q5T013 |
| IF2P_HUMAN | O60841 |
| IF5_HUMAN | P55010 |
| IGLL5_HUMAN | B9A064 |
| IL6RB_HUMAN | P40189 |
| ILF2_HUMAN | Q12905 |
| IMDH1_HUMAN | P20839 |
| IMDH2_HUMAN | P12268 |
| IPO9_HUMAN | Q96P70 |
| ITA1_HUMAN | P56199 |
| ITM2B_HUMAN | Q9Y287 |
| JHD2C_HUMAN | Q15652 |
| K319L_HUMAN | Q8IZA0 |
| KANL1_HUMAN | Q7Z3B3 |
| KAPCA_HUMAN | P17612 |
| KBL_HUMAN | O75600 |
| KPCA_HUMAN | P17252 |
| KTN1_HUMAN | Q86UP2 |
| KTNB1_HUMAN | Q9BVA0 |
| LIMK1_HUMAN | P53667 |
| LMBD2_HUMAN | Q68DH5 |
| LMBL3_HUMAN | Q96JM7 |
| LOXL2_HUMAN | Q9Y4K0 |
| LPIN1_HUMAN | Q14693 |
| LPPRC_HUMAN | P42704 |
| LRC41_HUMAN | Q15345 |
| LTN1_HUMAN | O94822 |
| LTOR3_HUMAN | Q9UHA4 |
| MAT2B_HUMAN | Q9NZL9 |
| MCM3_HUMAN | P25205 |
| MCU_HUMAN | Q8NE86 |
| METK1_HUMAN | Q00266 |
| MIC10_HUMAN | Q5TGZ0 |
| MINT_HUMAN | Q96T58 |
| MKRN2_HUMAN | Q9H000 |
| MON2_HUMAN | Q7Z3U7 |
| MPIP2_HUMAN | P30305 |
| MRP1_HUMAN | P33527 |
| MSPD2_HUMAN | Q8NHP6 |
| MTD2L_HUMAN | Q9H903 |
| MTMRA_HUMAN | Q9NXD2 |
| MTX2_HUMAN | O75431 |
| MVP_HUMAN | Q14764 |
| MYH9_HUMAN | P35579 |
| MYO1C_HUMAN | O00159 |
| NASP_HUMAN | P49321 |
| NBN_HUMAN | O60934 |
| NCOR2_HUMAN | Q9Y618 |
| NEBU_HUMAN | P20929 |
| NEP1_HUMAN | Q92979 |
| NEUL_HUMAN | Q9BYT8 |
| NEUR3_HUMAN | Q9UQ49 |
| NFS1_HUMAN | Q9Y697 |
| NHSL1_HUMAN | Q5SYE7 |
| NIBL1_HUMAN | Q96TA1 |
| NKTR_HUMAN | P30414 |
| NP1L1_HUMAN | P55209 |
| NPL4_HUMAN | Q8TAT6 |
| NQO1_HUMAN | P15559 |
| NSN5C_HUMAN | Q63ZY6 |
| NT5D3_HUMAN | Q86UY8 |
| NTH_HUMAN | P78549 |
| NU205_HUMAN | Q92621 |
| NUP43_HUMAN | Q8NFH3 |
| ODO1_HUMAN | Q02218 |
| P4HA1_HUMAN | P13674 |
| P5CS_HUMAN | P54886 |
| PA1B2_HUMAN | P68402 |
| PACS1_HUMAN | Q6VY07 |
| PARP2_HUMAN | Q9UGN5 |
| PCD12_HUMAN | Q9NPG4 |
| PCY1B_HUMAN | Q9Y5K3 |
| PDLI5_HUMAN | Q96HC4 |
| PDPR_HUMAN | Q8NCN5 |
| PGBM_HUMAN | P98160 |
| PHF19_HUMAN | Q5T6S3-2 |
| PHLP1_HUMAN | O60346 |
| PI3R4_HUMAN | Q99570 |
| PKHA5_HUMAN | Q9HAU0 |
| PLEC_HUMAN | Q15149 |
| PLST_HUMAN | P13797 |
| PP2BA_HUMAN | Q08209 |
| PRDX1_HUMAN | Q06830 |
| PRDX4_HUMAN | Q13162 |
| PRKRA_HUMAN | O75569 |
| PRP31_HUMAN | Q8WWY3 |
| PRP8_HUMAN | Q6P2Q9 |
| PRS7_HUMAN | P35998 |
| PSA7_HUMAN | O14818 |
| PSB3_HUMAN | P49720 |
| PSD12_HUMAN | O00232 |
| PTN2_HUMAN | P17706 |
| PTPRF_HUMAN | P10586 |
| PTPRG_HUMAN | P23470 |
| PURA2_HUMAN | P30520 |
| PUS10_HUMAN | Q3MIT2 |
| PYR1_HUMAN | P27708 |
| PYRG1_HUMAN | P17812 |
| Q5HYG5_HUMAN | Q5HYG5 |
| RAB14_HUMAN | P61106 |
| RAB9A_HUMAN | P51151 |
| RACK1_HUMAN | P63244 |
| RAD17_HUMAN | O75943 |
| RBP10_HUMAN | Q6VN20 |
| RBP2_HUMAN | P49792 |
| RCAN1_HUMAN | P53805 |
| RCC2_HUMAN | Q9P258 |
| RECQ1_HUMAN | P46063 |
| RHG01_HUMAN | Q07960 |
| RHG10_HUMAN | A1A4S6 |
| RHOQ_HUMAN | P17081 |
| RL10_HUMAN | P27635 |
| RM24_HUMAN | Q96A35 |
| RPB3_HUMAN | P19387 |
| RPC1_HUMAN | O14802 |
| RSSA_HUMAN | P08865 |
| RTCB_HUMAN | Q9Y310 |
| SAMH1_HUMAN | Q9Y3Z3 |
| SAP18_HUMAN | O00422 |
| SC22B_HUMAN | O75396 |
| SC24B_HUMAN | O95487 |
| SC24D_HUMAN | O94855 |
| SCFD2_HUMAN | Q8WU76 |
| SDE2_HUMAN | Q6IQ49 |
| SDHA_HUMAN | P31040 |
| SEM6C_HUMAN | Q9H3T2 |
| SEP11_HUMAN | Q9NVA2 |
| SEPT2_HUMAN | Q15019 |
| SEPT9_HUMAN | Q9UHD8 |
| SERPH_HUMAN | P50454 |
| SF3A1_HUMAN | Q15459 |
| SF3B1_HUMAN | O75533 |
| SHB_HUMAN | Q15464 |
| SIAE_HUMAN | Q9HAT2 |
| SIGIR HUMAN | Q6IA17 |
| SIN1_HUMAN | Q9BPZ7 |
| SLIT2_HUMAN | O94813-2 |
| SNR40_HUMAN | Q96DI7 |
| SNX6_HUMAN | Q9UNH7 |
| SNX9_HUMAN | Q9Y5X1 |
| SPAT5_HUMAN | Q8NB90 |
| SPOP_HUMAN | O43791 |
| SPRY4_HUMAN | Q8WW59 |
| SPS1_HUMAN | P49903 |
| SPTB1_HUMAN | P11277 |
| SPTN1_HUMAN | Q13813 |
| SQSTM_HUMAN | Q13501 |
| SRBD1_HUMAN | Q8N5C6 |
| SRP54_HUMAN | P61011 |
| SRRT_HUMAN | Q9BXP5 |
| STAR9_HUMAN | Q9P2P6 |
| STIP1_HUMAN | P31948 |
| SUFU_HUMAN | Q9UMX1 |
| SUMO2_HUMAN | P61956 |
| SYNE1_HUMAN | Q8NF91 |
| SYTL2_HUMAN | Q9HCH5-7 |
| SYTM_HUMAN | Q9BW92 |
| T126A_HUMAN | Q9H061 |
| T22D2_HUMAN | O75157 |
| TAU_HUMAN | P10636 |
| TBB2A_HUMAN | Q13885 |
| TBG1_HUMAN | P23258 |
| TCPG_HUMAN | P49368 |
| TCRG1_HUMAN | O14776 |
| TEX2_HUMAN | Q8IWB9 |
| TGFI1_HUMAN | O43294 |
| THOC6_HUMAN | Q86W42 |
| THOP1_HUMAN | P52888 |
| THRB_HUMAN | P00734 |
| TIF1B_HUMAN | Q13263 |
| TITIN_HUMAN | Q8WZ42 |
| TMED8_HUMAN | Q6PL24 |
| TNS2_HUMAN | Q63HR2 |
| TPC11_HUMAN | Q7Z392 |
| TPM1_HUMAN | P09493 |
| TRI25_HUMAN | Q14258 |
| TRIP4_HUMAN | Q15650 |
| TRM13_HUMAN | Q9NUP7 |
| TRRAP_HUMAN | Q9Y4A5 |
| TSC2_HUMAN | P49815 |
| TTC13_HUMAN | Q8NBP0 |
| TTC27_HUMAN | Q6P3X3 |
| TTC9C_HUMAN | Q8N5M4 |
| TXNL1_HUMAN | O43396 |
| U2AF1_HUMAN | Q01081 |
| U5S1_HUMAN | Q15029 |
| UBA1_HUMAN | P22314 |
| UBP15_HUMAN | Q9Y4E8 |
| UBP24_HUMAN | Q9UPU5 |
| UBP7_HUMAN | Q93009 |
| UGPA_HUMAN | Q16851 |
| URB2_HUMAN | Q14146 |
| UTP4_HUMAN | Q969X6 |
| VATB1_HUMAN | P15313 |
| VATH_HUMAN | Q9UI12 |
| VINC_HUMAN | P18206 |
| VPP1_HUMAN | Q93050 |
| WDR43_HUMAN | Q15061 |
| WDR7_HUMAN | Q9Y4E6 |
| XPO1_HUMAN | O14980 |
| XRN2_HUMAN | Q9H0D6 |
| YETS2_HUMAN | Q9ULM3 |
| ZCCHV_HUMAN | Q7Z2W4 |
| ZN185_HUMAN | O15231 |

The sequence motif for these peptides contained only D at P1 and a high prevalence of hydrophobic residues (L, V, I, F, and M) at P4, confirming that a majority of these proteolysis events are due to iCasp activity (FIG. 8B). Interestingly, proline was the third most abundant amino acid at P4, suggesting that human caspase-4, like mouse caspase-11, possesses a distinct substrate specificity from caspase-1 (Ramirez M L G, et al. J Biol Chem 2018 293(18):7058-7067). Furthermore, CJ11 enriched for peptides with significant diversity at P3 and H/S/T at P2, which confirmed previous in vitro work on caspase-4 specificity (Thornberry N A, et al. J Biol Chem 1997 272(29):17907-17911).

Overall, only a few of these putative substrates were previously identified caspase 1 substrates (namely, HNRPK, TIF1B, MCM3, and GSDMD), with the remainder representing new iCasp substrates (Agard N J, Maltby D, & Wells J A Mol Cell Proteomics 2010 9(5):880-893; Lamkanfi M, et al. Mol Cell Proteomics 2008 7(12):2350-2363). Beyond differences in caspase, this result suggested that differences in cell type or inflammasome complex (e.g., canonical versus non-canonical) lead to cleavage of unique substrate pools. Gene ontology (GO) enrichment analyses were performed using Protein ANalysis THrough Evolutionary Relationships (PANTHER) to better understand the cellular processes affected by caspase-4. Processes such as RNA splicing, regulation of gene silencing, and Golgi to endosome transport were enriched alongside cell compartments such as the spliceosomal complex (FIG. 8C). A majority of the proteins involved in RNA splicing physically interact and are components of the spliceosome, which suggested that the caspase-4 non-canonical inflammasome alters or inhibits RNA splicing (FIG. 8D).

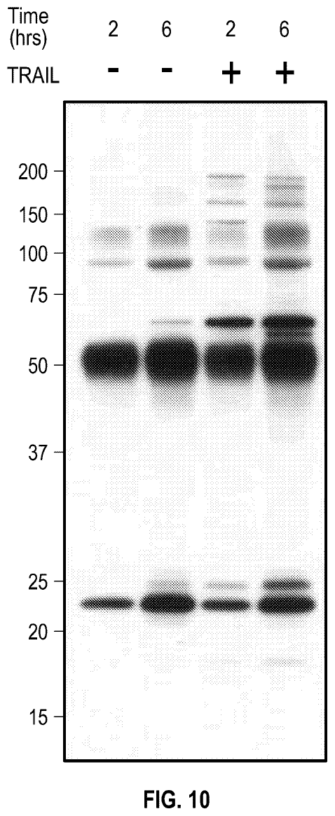

Notably, caspase-7 was cleaved under these experimental conditions, suggesting potential cross-talk between the non-canonical inflammasome and an apoptotic caspase. To confirm that this proteolysis event was due to caspase-4, CRISPR/Cas9 was used to knockout CASP4 in EA.hy926 cells. Upon stimulation with LPS, cleavage of both GSDMD and caspase-7 occurred in the wild-type EA.hy926, but no cleavage could be detected in the absence of caspase-4 (FIG. 8E). Furthermore, cleavage at Asp198 is known to activate caspase-7. Thus, the present results showed that activation of the non-canonical inflammasome can result in downstream activation of caspase-7 and likely eventual apoptosis in the absence of GSDMD-mediated pyroptosis. This finding agreed well with previous work on the canonical inflammasome and a caspase-11 genetic study (Tsuchiya K, et al. Nat Commun 2019 10(1):2091; Kang S J, Wang S, Kuida K, & Yuan J Cell Death Differ 2002 9(10):1115-1125). Since activation of caspase-7 occurs in this context, it is possible that some of the putative non-canonical inflammasome substrates are in fact cleaved by caspase-7 at a site in which P4 is not Asp. To evaluate whether CJ11 was capable of detecting such products, IP-Westerns were performed using CJ11 and lysates from EA.hy926 cell stimulated with TRAIL, a potent activator of the extrinsic apoptotic pathway TRAIL for 2 or 6 hours. Several cleavage products detected by CJ11 were observed, indicating that some substrates of the apoptotic caspases can be detected (FIG. 10). Given the short time scale of the LPS stimulation experiments (1 hour) and that activation of caspase-7 would occur after caspase-4 activation, without wishing to be bound by theory, it is anticipated that such substrates would be of lower abundance compared to those of the non-canonical inflammasome.

Overall, these results validate the utility of anti-cleaved iCaspase substrate antibodies such as CJ11 as an important tool to study the function of the non-canonical inflammasome and the iCasps in general.

1. An antibody that binds to a cleaved inflammatory caspase (iCaspase) substrate, wherein the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

2-10. (canceled)

11. An antibody that binds to a cleaved inflammatory caspase (iCaspase) substrate, wherein the antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid,
wherein the antibody comprises a variable heavy chain (VH) and a variable light chain (VL), wherein the antibody comprises:
a) a CDRH1, a CDRH2, and a CDRH3 of a VH chain comprising the amino acid sequence set forth in SEQ ID NO: 1 and a CDRL1, CDRL2, and CDRL3 of a VL chain comprising the amino acid sequence set forth in SEQ ID NO: 2, or
b) a CDRH1, a CDRH2, and a CDRH3 of a VH chain comprising the amino acid sequence set forth in SEQ ID NO: 9 and a CDRL1, CDRL2, and CDRL3 of a VL chain comprising the amino acid sequences set forth in SEQ ID NO: 10.

12. The antibody of claim 11, wherein the antibody comprises a CDRH1 amino acid sequence set forth in SEQ ID NO: 3; a CDRH2 amino acid sequence set forth in SEQ ID NO: 4; a CDRH3 set forth in SEQ ID NO:5; a CDRL1 amino acid sequence set forth in SEQ ID NO: 6; a CDRL2 amino acid sequence set forth in SEQ ID NO:7; and a CDRL3 amino acid sequence set forth in SEQ ID NO:8.

13. The antibody of claim 11, wherein the antibody comprises a VH chain amino acid set forth in SEQ ID NO: 1 and a VL chain amino acid set forth in SEQ ID NO:2.

14. (canceled)

15. The antibody of claim 11, wherein the antibody comprises a CDRH1 amino acid sequence set forth in SEQ ID NO: 11; a CDRH2 amino acid sequence set forth in SEQ ID NO: 12; a CDRH3 amino acid sequence set forth in SEQ ID NO:13; a CDRL1 amino acid sequence set forth in SEQ ID NO: 14; a CDRL2 amino acid sequence set forth in SEQ ID NO:15; and a CDRL3 amino acid sequence set forth in SEQ ID NO:16.

16. The antibody of claim 11, wherein the antibody comprises a VH chain amino acid sequence set forth in SEQ ID NO: 9 and a VL chain amino acid sequence set forth in SEQ ID NO:10.

17. Nucleic acid encoding the antibody of claim 11.

18. A host cell comprising the nucleic acid of claim 17.

19. A method of screening for an antibody that binds to a cleaved iCaspase substrate, wherein the antibody specifically binds to a peptide comprising the amino acid sequence P4-P3-P2-D at the C-terminus of the peptide, wherein the antibody does not bind to peptides comprising the amino acid sequence D-X-X-D at the C-terminus, wherein X is any amino acid, comprising
i) providing an antibody library;
ii) positively selecting antibodies that bind to a peptide comprising the amino acid sequence P4-P3-P2-D motif at the C-terminus; and
iii) negatively selecting antibodies that bind to a peptide comprising the amino acid sequence D-X-X-D at the C-terminus,
thereby producing an antibody that specifically binds to a peptide comprising the amino acid P4-P3-P2-D at the C-terminus, and does not bind to peptides comprising the amino acid sequence D-X-X-D at the C-terminus.

20. The method of claim 19, further comprising negatively selecting antibodies that bind to a peptide comprising the amino acid sequence E-X-X-D at the C-terminus.

21. The method of claim 20, wherein negatively selecting antibodies that bind to a peptide comprising the amino acid sequence E-X-X-D at the C-terminus is performed simultaneously with step iii).

22. The method of claim 20, wherein negatively selecting antibodies that bind to a peptide comprising the amino acid sequence E-X-X-D at the C-terminus is performed before or after step iii).

23. The method of claim 19, wherein P4 is a hydrophobic amino acid.

24. (canceled)

25. The method of claim 19, wherein the library is produced by immunizing a mammal with a peptide library comprising peptides comprising the following sequences W-P3-P2-D, Y-P3-P2-D, I-P3-P2-D, and L-P3-P2-D, wherein P3 is an equimolar mixture of E, V, and Q and P2 is an equimolar mixture of H, S, and T, wherein the mammal produces antibodies to the peptides.

26. (canceled)

27. The method of claim 19, wherein steps ii)-iii) are repeated two or more times.

28. An antibody produced by the method of claim 19.

29. A method of detecting cleavage of an iCaspase substrate in a sample comprising
i) contacting the sample with an anti-cleaved iCaspase substrate antibody, and
ii) detecting a cleaved iCaspase substrate
wherein the anti-cleaved iCaspase substrate antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

30-31. (canceled)

32. A method of enriching cleaved iCaspase substrates in a sample comprising a mixture of polypeptides
i) contacting the sample with an anti-cleaved iCaspase substrate antibody; and
ii) selecting antibody-bound polypeptides from the sample,
wherein the anti-cleaved iCaspase substrate antibody specifically binds to a peptide comprising the amino acid sequence P4-P3-P2-D at the C-terminus of the peptide, wherein the antibody does not bind to peptides comprising the amino acid sequence D-X-X-D or E-X-X-D at the C-terminus, wherein X is any amino acid.

33-34. (canceled)

35. A library of cleaved iCaspase substrates produced by the method of claim 32.

36. A kit for detecting a cleaved iCaspase substrate in a sample comprising an anti-cleaved iCaspase substrate antibody and instructions for use, wherein the anti-cleaved iCaspase substrate antibody specifically binds to a peptide comprising the amino acid sequence P4-P3-P2-D at the C-terminus of the peptide, wherein P4 is a hydrophobic amino acid, wherein the antibody does not bind to peptides comprising the amino acid sequence D-X-X-D at the C-terminus, wherein X is any amino acid.

37. (canceled)

38. The antibody of claim 11, wherein the antibody binds to a cleaved substrate of Caspase 1, Caspase 4, Caspase 5, or Caspase 11.

39. The antibody of claim 11, wherein the antibody is a rabbit, rodent, or goat antibody.

40. The antibody of claim 11, wherein the antibody is a full-length antibody, a Fab fragment, or an scFv.

41. The antibody of claim 11, wherein the antibody is conjugated to a label.
